(19) United States
(12) Patent Application Publication
(10) Pub. No.: US 2015/0147301 A1
(43) Pub. Date: May 28, 2015

(54) METHODS AND COMPOSITIONS OF PRODUCING PATIENT-SPECIFIC MULTIPOTENT NEURONAL STEM CELLS

(71) Applicant: INTERNATIONAL STEM CELL CORPORATION, Carlsbad, CA (US)

(72) Inventors: Ruslan Semechkin, Rancho Santa Fe, CA (US); Dmitry Isaev, Vista, CA (US)

(21) Appl. No.: 14/591,764

(22) Filed: Jan. 7, 2015

Related U.S. Application Data
(62) Division of application No. 13/396,560, filed on Feb. 14, 2012, now abandoned.
(60) Provisional application No. 61/442,711, filed on Feb. 14, 2011.

(51) Int. Cl. C12N 5/0797; A61K 35/30; C12N 5/0793

(52) U.S. Cl. CPC C12N 5/0623; C12N 5/0619; A61K 35/30; C12N 2500/70; C12N 2500/46; C12N 2500/32
USPC 424/93.7; 435/325; 435/368; 435/375

(57) ABSTRACT

The present invention relates to the seminal discovery of compositions and a method of producing NSC obtained from stem cells derived from parthenogenically activated human oocytes (phNSC). The phNSC of the invention maintain proliferative and differentiation potential during cultivation and expansion.

### RELATED APPLICATION DATA

This application is a divisional of U.S. application Ser. No. 13/396,560, filed Feb. 14, 2012, currently pending, which claims the benefit of priority under 35 U.S.C. §119(e) of the U.S. Patent No. 61/442,711, filed Feb. 14, 2011, the entire content of which is incorporated herein by reference.

### BACKGROUND OF THE INVENTION

1. Field of the Invention

The invention relates generally to stems cells, and more specifically to a method and compositions for producing neuronal stem cells using human stem cells.

2. Background Information

Human embryonic stem cells (hESC) cells are pluripotent cells that can differentiate into an array of cell types. When injected into immune-deficient mice, embryonic stem cells form differentiated tumors (teratomas). However, embryonic stem cells that are induced in vitro to form embryoid bodies (EBs) provide a source of embryonic stem cell lines that are amenable to differentiation into multiple cell types characteristic of several tissues under certain growth conditions. For example, hESC have been differentiated into endoderm, ectoderm, and mesoderm derivatives.

Human ES cells and their differentiated progeny are important sources of human cells for therapeutic transplantation and for drug testing and development. Required by both of these goals is the provision of sufficient cells that are differentiated into tissue types suitable for a patient's needs or the appropriate pharmacological test. Associated with this is a need for an efficient and reliable method of producing differentiated cells from embryonic stem cells.

Parthenogenic activation of mammalian oocytes may be used to prepare oocytes for embryonic stem cell generation. Parthenogenic activation is the production of embryonic cells from a female gamete in the absence of any contribution from a male gamete.

Currently, a focus of stem cell research is the development of artificial organs, rehabilitation devices, or prosthesis to replace natural body tissues. This development generally envisages the use of biocompatible materials for engineering stem cells to control expansion/differentiation; i.e., the use of 3-D scaffolds (e.g., PLG scaffolds, chitosan scaffolds, PCL/PEG scaffolds) to create devices which mimic tissue-like function by providing mechanical support for proliferation.

Alternatively, transplantation of cultured stem cells or differentiated stem cells is envisioned as a therapeutic modality. These methods are generally known as in vivo tissue engineering or in situ generation. While much of the work in this area purports the direct transplantation of cultured cells, as a practical matter, such modalities often require seeding differentiated stem cells within porous scaffold biomaterials (e.g., cardiomyocytes derived from stem cells and gels or porous alginate).

Unfertilized human oocytes can be artificially activated by appropriate chemical stimuli to develop into parthenogenetic blastocysts. The inner cell mass of such blastocysts can be isolated and expanded as stem cell lines. First intentionally obtained by Revazova et al., human parthenogenetic stem cells (hpSC) are similar to human embryonic stem cells (hESC) in their proliferation capacity and multilineage in vitro differentiation [1, 2]. The hpSC can be either heterozygous or homozygous depending on the way the genome forms from only the maternal chromosome set. Homozygous hpSC may be useful as a source of cells for transplantations since the set of HLA genes in hpSC is able to produce differentiated derivatives less susceptible to immune rejection. Furthermore, if the HLA type is common, differentiated derivatives will match many millions of individuals [2, 3]. In addition to these Immunogenetics advantages, as parthenogenesis does not involve the destruction of a viable human embryo, the use of hpSC does not raise the same ethical concerns as conventional hESC. Thus, hpSC are an attractive alternative to other pluripotent stem cells as a source of somatic cell lines, including the multipotent neural stem cells (NSC).

NSC are self-renewing multipotent stem cells of nervous system, which have the capacity to differentiate into neurons, oligodendrocytes and astrocytes [4]. NSC can be obtained directly from fetal and adult central nervous system or by mean of induced neural differentiation from pluripotent stem cells. Obtained as a cell culture NSC are able to proliferate in vitro without losing their capacity for differentiation for a relatively long time, and hence provide reserve of cell material for further applications. NSC are considered as a perspective remedy for recovery therapy of neurodegenerative diseases, such as Alzheimer's disease, Parkinson's disease, Huntington's disease etc., as well as for spinal cord injuries leading to immobility. Successful experiments with animal models confirm efficiency of cell therapy with usage of NSC [5].

The capacity to differentiate into neurons and glial cells was experimentally proved for mice [6], primate [7] and human parthenogenetic stem cells [1, 2, 8]. Parthenogenetic stem cells bear two sets of maternally imprinted genes, which were assumed to be the obstacle for the differentiation into derivatives of all three germ layers. However, experiments with chimeric animals revealed the less degree of parthenogenetic cells elimination in the tissues and organs of ectodermal origin including neural system [9]. Cibelli et al. described the establishing of non-human primate Macaca fascicularis parthenogenetic stem cells, this cell line was called Cyno-1 [7]. As a proof of pluripotent state, Cyno-1 in vitro differentiation was performed, and neural derivatives were obtained among others. Later, Sânchez-Pernaute et al. obtained dopamine neurons from Cyno-1 in vitro by means of directed differentiation, and showed their effective therapy for rat and monkey Parkinson's disease model [10]. Neural differentiation of phSC in vitro was shown by Revazova et al. [1, 2] and Harness et al. [8]. Despite of these studies, long proliferating human parthenogenetic NSC still have not been obtained.

### SUMMARY OF THE INVENTION

The present invention relates to the seminal discovery of compositions and methods of producing NSC obtained from stem cells derived from parthenogenically activated human oocytes (phNSC). The phNSC of the invention maintain proliferative and differentiation potential during cultivation and expansion.

In one embodiment, the invention provides for an isolated neuronal stem cell, which is differentiated from a parthenogenetically activated oocyte. In another aspect the neuronal stem cells are histocompatible with the oocyte donor. In an additional aspect, the neuronal stem cell has a different pattern of zygosity from an ESC. In another aspect, the neuronal stem cell contains only the maternal genome. In one aspect the neuronal stem cell is histocompatible with the oocyte donor, has a different pattern of zygosity from an ESC and contains only the maternal genome. In an additional aspect the neuronal stem cell is histocompatible with a population group based on a matching haplotype. In a further aspect, the neuronal stem cells are transplantable to humans. In an additional aspect, the neuronal stem cells are undifferentiated, partially differentiated or fully differentiated. In a further aspect, the neuronal stem cell can be differentiated into a neuronal cell. The neuronal stem cells can be differentiated into a neuronal cell selected from the group consisting of a neuron, a glial cell, an oligodendrocyte and an astrocyte. In one aspect the differentiated neuronal cell is a neuron. In a further aspect the neuron is selected from the group consisting of a cholinergic neuron, a GABAergic neuron, a glutamatergic neuron, a dopaminergic neuron and a serotonergic neuron. The differentiated neuronal cell is histocompatible with the oocyte donor, has a different pattern of zygosity from an ESC and contains only the maternal genome. In a further aspect the neuronal stem cell expresses neural markers selected from the group consisting of SOX2, Nestin, Mushashi-1, TUBB3, MAP2, FOXO4, GFAP, CD113 and CD15.

In another embodiment, the invention provides a method for producing a neuronal stem cells by differentiating parthenogenetically derived human stem cells by a) growing parthenogenetically derived human stem cells on a feeder layer of fibroblast cells for at least 2 days; b) growing parthenogenetically derived human stem cells on a petri dish without fibroblast feeder layer for at least 1 day; c) culturing the cells in a neuronal induction media; d) obtaining a single cell suspension of the cells from (c); and e) culturing the single cells from step (d) on a petri dish with no fibroblast feeder layer in a neuronal proliferation media. In one aspect the neuronal induction media is made of Penicillin-Streptomycin-Amphotericin Solution (VWR, Radnor Pa.), DMEM/F12 (Invitrogen Grand Island, N.Y.), L-Glutamine (Invitrogen Grand Island, N.Y.), MEM Non-Essential Amino Acids Solution (Invitrogen Grand Island, N.Y.), N2 Supplement (Invitrogen Grand Island, N.Y.); and bFGF (Peprotech Rocky Hill, N.J.). In a further aspect L-Glutamine is present at 2 mM, MEM Non-Essential Amino Acids Solution is present at 0.1 mM and bFGF is present at 4-20 ng/ml in the neuronal induction media. In another aspect, the neuronal proliferation media is made of Penicillin-Streptomycin-Amphotericin Solution (VWR, Radnor Pa.), DMEM/F12 (Invitrogen Grand Island, N.Y.), GlutaMAX™-I (Invitrogen Grand Island, N.Y.), StemPro® Neural Supplement (Invitrogen Grand Island, N.Y.), 20 ng/ml bFGF (Peprotech Rocky Hill, N.J.) and 20 ng/ml EGF (Invitrogen Grand Island, N.Y.). In an additional aspect, FGF and EGF are present at 20 ng/ml in the neuronal proliferation media. In a further aspect, the petri dish is coated with CELLstart™ (Invitrogen Grand Island, N.Y.). The invention also provides for a neuronal stem cell produced by this method. In an additional aspect, a neuroepithelial rosette forms in about 1-2 weeks of culture in the neuronal induction media.

In a further embodiment, the invention provides for isolated neuronal stem cells derived from parthenogenetically derived human stem cells by a) growing parthenogenetically derived human stem cells on a feeder layer of fibroblast cells for at least 2 days; b) growing parthenogenetically derived human stem cells on a petri dish with no fibroblast feeder layer for at least 1 day; c) culturing the cells in a neuronal induction media; d) obtaining a single cell suspension of the cells from (c); and e) culturing the single cells from step (d) on a petri dish with no fibroblast feeder layer in a neuronal proliferation media. In one aspect, the neuronal stem cells express neural markers selected from the group consisting of: SOX2, Nestin, Mushashi-1, TUBB3, MAP2, FOXO4, GFAP, CD113 and CD15. In another aspect, the neuronal stem cells maintain the neuronal phenotype for at least 27 passages. In another aspect the neuronal stem cells are histocompatible with the oocyte donor. In an additional aspect, the neuronal stem cell has a different pattern of zygosity from an ESC. In another aspect, the neuronal stem cell contains only the maternal genome. In a further aspect, the neuronal stem cells are transplantable to humans. In an additional aspect, the neuronal stem cells are undifferentiated, partially differentiated or fully differentiated. In a further aspect, the neuronal stem cells can be differentiated into neuronal cells. In another aspect, the neuronal cells differentiated from neuronal stem cells can be neurons, glial cells, oligodendrocytes and astrocytes. In one aspect the differentiated neuronal cell is a neuron. In a further aspect the neuron is selected from the group consisting of a cholinergic neuron, a GABAergic neuron, a glutamatergic neuron, a dopaminergic neuron and a serotonergic neuron.

In one embodiment, the invention provides a method of treating a neurologic disorder using neuronal stem cells produced from parthenogenetically derived from oocytes. In one aspect, the neurologic disorder is selected from the group consisting of epilepsy, convulsions, neurotoxic injury, hypoxia, anoxia, ischemia, stroke, cerebrovascular accident, brain or spinal cord trauma, myocardial infarct, physical trauma, drowning, suffocation, perinatal asphyxia, hypoglycemic events, neurodegeneration, Alzheimer's disease, senile dementia, Amyotrophic Lateral Sclerosis, Multiple Sclerosis, Parkinson's disease, Huntington's disease, Down's Syndrome, Korsakoff's disease, schizophrenia, AIDS dementia, multi-infarct dementia, Binswanger dementia, neuronal damage, seizures, chemical toxicity, addiction, morphine tolerance, opiate tolerance, opioid tolerance, barbiturate tolerance, acute and chronic pain, migraine, anxiety, major depression, manic-depressive illness, obsessive-compulsive disorder, schizophrenia and mood disorders, bipolar disorder, unipolar depression, dysthymia, seasonal effective disorder, dystonia or other movement disorders, sleep disorder, muscle relaxation and urinary incontinence. In a further aspect, the neuronal stem cells are implanted into a patient in need of such treatment.

In a further embodiment, the invention provides a method of differentiating neuronal stem cells by culturing neuronal stem cells in neuronal differentiation media. In one aspect, the neuronal differentiation media contains Penicillin-Streptomycin-Amphotericin (VWR Radnor, Pa.); DMEM/F12 (Invitrogen Grand Island, N.Y.); GlutaMAX™-I (Invitrogen Grand Island, N.Y.); and StemPro® Neural Supplement (Invitrogen Grand Island, N.Y.). In a further aspect, the neuronal stem cells are differentiated into a neuronal cell selected from the group consisting of a neuron, a glial cell, an oligodendrocyte and an astrocyte. The invention also provides for the neuronal cells differentiated from the neuronal stem cells. In one aspect, the neuronal stem cells are produced from parthenogenetically derived human stem cells. In another aspect the neuronal stem cells are histocompatible with the oocyte donor. In an additional aspect, the neuronal stem cell has a different pattern of zygosity from an ESC. In another aspect, the neuronal stem cell contains only the maternal genome. In a further aspect, the neuronal stem cells are transplantable to humans. In an additional aspect, the neuronal stem cells are undifferentiated, partially differentiated or fully differentiated. In one aspect the differentiated neuronal cell is a neuron. In a further aspect the neuron is selected from the group consisting of a cholinergic neuron, a GABAergic neuron, a glutamatergic neuron, a dopaminergic neuron and a serotonergic neuron. In one aspect the differentiated neuronal cell is a neuron. In a further aspect the neuron is selected from the group consisting of a cholinergic neuron, a GABAergic neuron, a glutamatergic neuron, a dopaminergic neuron and a serotonergic neuron.

In one embodiment, the invention provides for a method for producing neuronal stem cells by differentiating parthenogenetically derived human stem cells by: a) cultivation of human pluripotent stem cells in feeder-free conditions; b) exposure of said cells to neuronal induction medium; c) mechanical isolation of partially differentiated cells; and d) further expansion and maintenance of said cells until maturation. In one aspect the neuronal induction media comprises: a) Penicillin-Streptomycin-Amphotericin Solution; b) DMEM/F12; c) MEM Non-Essential Amino Acids Solution; d) L-Glutamine; e) N2 Supplement; and f) bFGF. In a further aspect L-Glutamine is present at 2 mM, MEM Non-Essential Amino Acids Solution is present at 0.1 mM and bFGF is present at 4-20 ng/ml in the neuronal induction media. In another aspect the neuronal proliferation media comprises: a) Penicillin-Streptomycin-Amphotericin; b) DMEM/F12; c) GlutaMAX™-I; d) StemPro® Neural Supplement; e) bFGF; and f) EGF. In an additional aspect, FGF and EGF are present at 20 ng/ml in the neuronal proliferation media. In an additional aspect the feeder-free conditions utilize the ECM substrate including but not limited to: CELLstart, Matrigel, laminin, gelatin, fibronectin. The invention also provides for the neuronal stem cell produced by the method. In a further aspect, a neuroepithelial rosette forms after 1-2 weeks.

In an additional embodiment, the invention provides for isolated neuronal stem cells derived from parthenogenetically derived human stem cells using the method comprising: a) cultivation of human pluripotent stem cells in feeder-free conditions; b) exposure of said cells to neuronal induction medium; c) mechanical isolation of partially differentiated cells; and d) further expansion and maintenance of said cells until maturation. In one aspect, the cells express neural stem cell markers selected from the group consisting of: SOXB1-family NES, MSH-1, CXCR4, CCND1, LHX2, PAX6, GAP43. In a further aspect the neuronal stem cells maintain the neuronal phenotype for at least 30 passages. In one aspect, the neuronal stem cells can differentiate into neuronal cells. In an additional aspect, the neuronal cells are selected from the group consisting of neurons, astrocytes and oligodendrocytes. In one aspect the differentiated neuronal cell is a neuron. In a further aspect the neuron is selected from the group consisting of a cholinergic neuron, a GABAergic neuron, a glutamatergic neuron, a dopaminergic neuron and a serotonergic neuron.

In a further embodiment, the invention provides a method of treating a neurologic disorder using neuronal stem cells derived from parthenogenetically derived from oocytes. In one aspect the neurologic disorder is selected from the group consisting of: epilepsy, convulsions, neurotoxic injury, ischemia, stroke, cerebrovascular accident, brain or spinal cord trauma, physical trauma, Alzheimer's disease, senile dementia, Amyotrophic Lateral Sclerosis, Multiple Sclerosis, Parkinson's disease, Huntington's disease, schizophrenia, neuronal damage, migraine, anxiety, major depression, manic-depressive illness, obsessive-compulsive disorder, schizophrenia and mood disorders, bipolar disorder, unipolar depression, dystonia or other movement disorders, sleep disorder, muscle relaxation. In a further aspect, the neuronal stem cells are implanted into a patient in need of such treatment.

In one embodiment, the invention provides for a method of differentiating neuronal stem cells, the method comprising culturing neuronal stem cells in neuronal differentiation media. In one aspect the neuronal differentiation media comprises: a) Penicillin-Streptomycin-Amphotericin; b) DMEM/F12; c) GlutaMAX™-I; and d) StemPro® Neural Supplement. In an additional aspect, the neuronal stem cells are differentiated into a neuronal cell selected from the group consisting of: a neuron, an oligodendrocyte and an astrocyte. In one aspect the differentiated neuronal cell is a neuron. In a further aspect the neuron is selected from the group consisting of a cholinergic neuron, a GABAergic neuron, a glutamatergic neuron, a dopaminergic neuron and a serotonergic neuron. The Invention also provides for the differentiated cells produced by this method. In one aspect, the cells are differentiated from a parthenogenetically activated oocyte.

### BRIEF DESCRIPTION OF THE DRAWINGS

FIG. 1 is a graph showing the relative gene expression in hpSC (dark bars) and in the NEP rosettes (grey bars) on the 7^{th} day of neural induction.

FIG. 2 is a graph showing the relative transcriptional activity levels of important genes in phNSC (dark bars) and in hNSC H9 (grey bars).

FIG. 3 is a graph showing neuronal markers TUBB3 and MAP2 and glial markers GFAP and FOXO4 expression in spontaneously differentiated phNSC (dark bars) and hNSC (grey bars).

FIG. 4 is a graph showing that the parthenogenetically derived dopaminergic neurons are capable of firing an action potential,

### DETAILED DESCRIPTION OF THE INVENTION

The present invention relates to the seminal discovery of compositions and a method of producing NSC obtained from stem cells derived from parthenogenically activated human oocytes (phNSC). The phNSC of the invention maintain proliferative and differentiation potential during cultivation and expansion.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

“Differentiation” refers to a change that occurs in cells to cause those cells to assume certain specialized functions and to lose the ability to change into certain other specialized functional units. Cells capable of differentiation may be any of totipotent, pluripotent or multipotent cells. Differentiation may be partial or complete with respect to mature adult cells.

“Parthenogenesis” (“parthenogenically activated” and “parthenogenetically activated” is used interchangeably) the process by which activation of the oocyte occurs in the absence of sperm penetration, and refers to the development of an early stage embryo comprising trophectoderm and inner cell mass that is obtained by activation of an oocyte or embryonic cell, e.g., blastomere, comprising DNA of all female origin. In a related aspect, a “parthenote” refers to the resulting cell obtained by such activation. In another related aspect, “blastocyst: refers to a cleavage stage of a fertilized of activated oocyte comprising a hollow ball of cells made of outer trophoblast cells and an inner cell mass (ICM). In a further related aspect, “blastocyst formation” refers to the process, after oocyte fertilization or activation, where the oocyte is subsequently cultured in media for a time to enable it to develop into a hollow ball of cells made of outer trophoblast cells and ICM (e.g., 5 to 6 days).

A parthenote genome can contain a single or double set of epigenetically imprinted maternal chromosomes. There is no paternal genome and consequently, the “parthenogenetic blastocyst-like structures do not possess a functional genome that can be considered distinctive of a human embryo” [17]. As a result of the paternal DNA's influence on the development of the extraembryonic tissues, in the absence of paternal DNA mammalian eggs are incapable of progressing through the stages of natural embryogenesis . . . . ” [18]. Further, parthenotes are not totipotent [17]. Further, the process of becoming a human being requires both maternal and paternal DNA imprinting. Because parthenotes contain only the maternal genome, parthenogenic activation clearly does not involve paternal DNA imprinting and thus is a different process, leading to a different organism. The lack of proper DNA imprinting can be measured both on a molecular level (at the blstocyte stage) and on a macroscopic level by observing the lack of proper extra-embryonic tissues (i.e. the placenta).

Additionally, Parthenogenic stem cells differ in several important aspects from stems cells derived using other methods, including nuclear transfer. First, parthenogenetically derived stem cells are histocompatible with the oocyte donor. Parthenogenetically derived stem cells provide an exact match to the oocyte's genome, both nuclear and mitochondrial. Stem cells derived by nuclear transfer may provide a nearly exact match to the nuclear donor's immune identity, matching nuclear but not mitochondrial genes. Second, parthenogenetically derived stem cells have a unique pattern of zygosity reflected by the distribution of heterosygosity in Single Nucleotide Polymorphisms in genomic DNA in condensed chromosomes evident during meiotic and mitotic divisions. Parthenotes exhibit unique patterns of zygosity of the genome found in the areas surrounding the centromere and the distal ends of the DNA as compared to stem cells (and their derivatives) derived from fertilized embryos, from adult stem cells or nuclear transfer cells. As shown in Kim et al [19], parthenogenetic cells retain pericentromeric homozygosity but show distal regions of heterozygosity (reflecting the failure of independent segregation of sister chromatids during meiosis II in the oocyte) or retain pericentromeric heterozygosity of genetic markers and have characteristic distal regions of homozygosity (reflecting the failure of segregation homologous sets of chromosomes during meiosis I in the oocyte the of the paternal chromosomes). These patterns of homozygosity and heterozygosity around the centromeres and distal ends of the chromosomes distinguish parthenogenetic stem cells and their derivatives from cells derived from fertilized embryos or cells derived from stem cells derived from fertilized embryos which demonstrate heterozygosity throughout the entire lengths of the chromosome. Third, parthenogenetically derived stem cells only contain the maternal DNA. Normal mammalian development requires contributions from both maternal and paternal chromosomes. Parthenotes are derived exclusively from activated oocytes. A parthenote genome (one produced through parthenogenesis) contains essentially either a single or double set of epigenetically imprinted maternal chromosomes, depending on whether the expulsion of chromosomes in a polar body which an oocyte attempts to emit after activation is permitted or suppressed, respectively. Parthenotes contain only the maternal chromosome, thus there is no paternal genome or other added genetic material. Fourth, parthenogenetically derived stem cells will always have the maternal karyotype, XX.

Furthermore, a parthenote contains only a single pronucleus. The single pronucleus contains only half of the genetic material required for fertilization, i.e. the maternal genome. Further unlike somatic cell nuclear transfer, which uses biological material that was derived from a process that commenced, and succeeded, in becoming a human being, a parthenote never uses biological material that was ever in the process of becoming a human being.

“Pluripotent cell” refers to a cell derived from an embryo produced by activation of a cell containing DNA of all female or male origin that can be maintained in vitro for prolonged, theoretically indefinite period of time in an undifferentiated state, that can give rise to different differentiated tissue types, i.e., ectoderm, mesoderm, and endoderm. The pluripotent state of the cells is preferably maintained by culturing inner cell mass or cells derived from the inner cell mass of an embryo produced by androgenetic or gynogenetic methods under appropriate conditions, for example, by culturing on a fibroblast feeder layer or another feeder layer or culture that includes leukemia inhibitory factor (LIF). The pluripotent state of such cultured cells can be confirmed by various methods, e.g., (i) confirming the expression of markers characteristic of pluripotent cells; (ii) production of chimeric animals that contain cells that express the genotype of the pluripotent cells; (iii) injection of cells into animals, e.g., SCID mice, with the production of different differentiated cell types in vivo; and (iv) observation of the differentiation of the cells (e.g., when cultured in the absence of feeder layer or LIF) into embryoid bodies and other differentiated cell types in vitro.

As used herein, “multipotent” or “multipotent cell” refers to a cell type that can give rise to a limited number of other particular cell types. As described above, definitive endoderm cells do not differentiate into tissues produced from ectoderm or mesoderm, but rather, differentiate into the gut tube as well as organs that are derived from the gut tube. In one embodiment, the definitive endoderm cells are derived from hESCs. Such processes can provide the basis for efficient production of human endodermal derived tissues such as pancreas, liver, lung, stomach, intestine and thyroid. For example, production of definitive endoderm may be the first step in differentiation of a stem cell to a functional insulin-producing β-cell. To obtain useful quantities of insulin-producing β-cells, high efficiency of differentiation is desirable for each of the differentiation steps that occur prior to reaching the pancreatic islet/β-cell fate. Since differentiation of stem cells to definitive endoderm cells represents perhaps the earliest step towards the production of functional pancreatic islet/β-cells, high efficiency of differentiation at this step is particularly desirable.

“Diploid cell” refers to a cell, e.g., an oocyte or blastomere, having a diploid DNA content of all male or female origin.

Haploid cell” refers to a cell, e.g., an oocyte or blastomere, having a haploid DNA content, where the haploid DNA is of all male or female origin.

“Activation” refers to a process where a fertilized or unfertilized oocyte, for example, but not limited to, in metaphase II of meiosis, undergoes a process typically including separation of the chromatid pairs, extrusion of the second polar body, resulting in an oocyte having a haploid number of chromosomes, each with one chromatid. Activation includes methods whereby a cell containing DNA of all male or female origin is induced to develop into an embryo that has a discernible inner cell mass and trophectoderm, which is useful for producing pluripotent cells but which is itself is likely to be incapable of developing into a viable offspring. Activation may be carried out, for example, under one of the following conditions: (1) conditions that do not cause second polar body extrusion; (ii) conditions that cause polar body extrusion but where the polar body extrusion is inhibited; or (iii) conditions that inhibit first cell division of the haploid oocyte.

“Metaphase II” refers to a stage of cell development where the DNA content of a cell consists of a haploid number of chromosomes with each chromosome represented by two chromatids.

In one embodiment, metaphase II oocytes are activated/cultured by incubating oocytes under various O₂ tension gas environments. In a related aspect, the low O₂ tension gas environment is created by a gas mixture comprising an O₂ concentration of about 2%, 3%, 4%, or 5%. In a further related aspect, the gas mixture comprises about 5% CO₂. Further, the gas mixture comprises about 90% N₂, 91% N₂, or 93% N₂. This gas mixture is to be distinguished from 5% CO₂ air, which is approximately about 5% CO₂, 20% O₂, and 75% N₂.

“O₂ tension” refers to the partial pressure (pressure exerted by a single component of a gas mixture) of oxygen in a fluid (i.e., liquid or gas). Low tension is when the partial pressure of oxygen (pO₂) is low and high tension is when the pO₂ is high.

“Defined-medium conditions” refer to environments for culturing cells where the concentration of components therein required for optimal growth are detailed. For example, depending on the use of the cells (e.g., therapeutic applications), removing cells from conditions that contain xenogenic proteins is important; i.e., the culture conditions are animal-free conditions or free of non-human animal proteins. In a related aspect, “in vitro fertilization (IVF) media” refers to a nutrient system which contains chemically defined substances on or in which fertilized oocytes can be grown.

“Extracellular matrix (ECM) substrates” refer to a surface beneath cells which supports optimum growth. For example, such ECM substrates include, but are not limited to, Matrigel, laminin, gelatin, and fibronectin substrates. In a related aspect, such substrates may comprise collagen IV, entactin, heparin sulfate proteoglycan, to include various growth factors (e.g., bFGF, epidermal growth factor, insulin-like growth factor-1, platelet derived growth factor, nerve growth factor, and TGF-β-1).

“Embryo” refers to an embryo that results upon activation of a cell, e.g., oocyte or other embryonic cells containing DNA of all male or female origin, which optionally may be modified, that comprises a discernible trophectoderm and inner cell mass, which cannot give rise to a viable offspring and where the DNA is of all male or female origin. The inner cell mass or cells contained therein are useful for the production of pluripotent cells as defined previously.

“Inner cell mass (ICM)” refers to the inner portion of an embryo which gives rise to fetal tissues. Herein, these cells are used to provide a continuous source of pluripotent cells in vitro. Further, the ICM includes the inner portion of the embryo that results from androgenesis or gynogenesis, i.e., embryos that result upon activation of cells containing DNA of all male or female origin. Such DNA, for example, will be human DNA, e.g., human oocyte or spermatozoal DNA, which may or may not have been genetically modified.

“Trophectoderm” refers to another portion of early stage embryo which gives rise to placental tissues, including that tissue of an embryo that results from androgenesis or gynogenesis, i.e., embryos that result from activation of cells that contain DNA of all male or female origin, e.g., human ovarian or spermatozoan.

“Differentiated cell” refers to a non-embryonic cell that possesses a particular differentiated, i.e., non-embryonic, state. The three earliest differentiated cell types are endoderm, mesoderm, and ectoderm.

“Substantially identical” refers to a quality of sameness regarding a particular characteristic that is so close as to be essentially the same within the ability to measure difference (e.g., by HLA typing, SNP analysis, and the like).

“Histocompatible” refers to the extent to which an organism will tolerate a graft of a foreign tissue.

In another embodiment, stem cells are generated from a parthogenetically activated human oocyte. In one aspect, a neuronal stem cell is obtained from a neuronal stem cell differentiated from stem cells derived from a parthenogenetically activated human oocyte.

In the native environment, immature oocytes (eggs) from the ovary undergo a process of maturation which results in the progression through meiosis to metaphase II of meiosis. The oocytes then arrest at metaphase II. In metaphase II, the DNA content of the cell consists of a haploid number of chromosomes, each represented by two chromatids.

The parthenogenetically activated oocytes, blastocysts, ICM, and autologous stem cells can be stored or “banked” in a manner that allows the cells to be revived as needed in the future. An aliquot of the parthenogenetically activated oocytes and autologous stem cells can be removed at any time, to be grown into cultures of many undifferentiated cells and then differentiated into a particular cell type or tissue type, and may then be used to treat a disease or to replace malfunctioning tissues in a subject. Since the cells are parthenogenetically derived from the donor, the cells can be stored so that an individual or close relative can have access to cells for an extended period of time.

In one embodiment, a cell bank is provided for storing parthenogenetically activated oocytes, blastocysts, ICM, and/or autologous stem cell samples. In another embodiment, methods for administering such a cell bank are provided. U.S. Published Patent Application No. 20030215942, which is incorporated by reference herein in its entirety, provides an example of a stem cell bank system.

In one embodiment, the invention provides for an isolated neuronal stem cell, which is differentiated from a parthenogenetically activated oocyte. In another aspect the neuronal stem cells are histocompatible with the oocyte donor. In an additional aspect, the neuronal stem cell has a different pattern of zygosity from an ESC. In another aspect, the neuronal stem cell contains only the maternal genome. In one aspect the neuronal stem cell is histocompatible with the oocyte donor, has a different pattern of zygosity from an ESC and contains only the maternal genome. In an additional aspect the neuronal stem cell is histocompatible with a population group based on a matching haplotype. In a further aspect, the neuronal stem cells are transplantable to humans. In an additional aspect, the neuronal stem cells are undifferentiated, partially differentiated or fully differentiated. In a further aspect, the neuronal stem cell can be differentiated into a neuronal cell. The neuronal stem cells can be differentiated into a neuronal cell selected from the group consisting of a neuron, a glial cell, an oligodendrocyte and an astrocyte. In one aspect the differentiated neuronal cell is a neuron. In a further aspect the neuron is selected from the group consisting of a cholinergic neuron, a GABAergic neuron, a glutamatergic neuron, a dopaminergic neuron and a serotonergic neuron. The differentiated neuronal cell is histocompatible with the oocyte donor, has a different pattern of zygosity from an ESC and contains only the maternal genome. In a further aspect the neuronal stem cell expresses neural markers selected from the group consisting of SOX2, Nestin, Mushashi-1, TUBB3, MAP2, FOXO4, GFAP, CD113 and CD15.

“Neuronal cells” refers to any cell associated with the brain, spine or any other part of the central nervous system. Neuronal cells include, but are not limited to, neurons, astrocytes, glial cells and oligodencrocytes.

A neuron is an electrically excitable cell that processes and transmits information by electrical and chemical signaling. Chemical signaling occurs via synapses, specialized connections with other cells. Neurons connect to each other to form neural networks. Neurons are the core components of the nervous system, which includes the brain, spinal cord, and peripheral ganglia. A number of specialized types of neurons exist: sensory neurons respond to touch, sound, light and numerous other stimuli affecting cells of the sensory organs that then send signals to the spinal cord and brain. Motor neurons receive signals from the brain and spinal cord, cause muscle contractions, and affect glands. Interneurons connect neurons to other neurons within the same region of the brain or spinal cord.

Neurons differ in the type of neurotransmitter hey manufacture. Some examples are:

Cholinergic neurons manufacture acetylcholine. Acetylcholine is released from presynaptic neurons into the synaptic cleft. It acts as a ligand for both ligand-gated ion channels and metabotropic (GPCRs) muscarinic receptors. Nicotinic receptors, are pentameric ligand-gated ion channels composed of alpha and beta subunits that bind nicotine. Ligand binding opens the channel causing influx of Na⁺ depolarization and increases the probability of presynaptic neurotransmitter release.

GABAergic neurons manufacture gamma aminobutyric acid (GABA). GABA is one of two neuroinhibitors in the CNS, the other being Glycine. GABA has a homologous function to ACh, gating anion channels that allow Cl— ions to enter the post synaptic neuron. Cl— causes hyperpolarization within the neuron, decreasing the probability of an action potential firing as the voltage becomes more negative.

Glutamatergic neurons manufactures glutamate. Glutamate is one of two primary excitatory amino acids, the other being Aspartate. Glutamate receptors are one of four categories, three of which are ligand-gated ion channels and one of which is a G-protein coupled receptor (often referred to as GPCR). AMPA and Kainate receptors both function as cation channels permeable to Na⁺ cation channels mediating fast excitatory synaptic transmission. NMDA receptors are another cation channel that is more permeable to Ca²⁺. The function of NMDA receptors is dependant on Glycine receptor binding as a co-agonist within the channel pore. NMDA receptors do not function without both ligands present. Metabotropic receptors, GPCRs modulate synaptic transmission and postsynaptic excitability. Glutamate can cause excitotoxicity when blood flow to the brain is interrupted, resulting in brain damage. When blood flow is suppressed, glutamate is released from presynaptic neurons causing NMDA and AMPA receptor activation more so than would normally be the case outside of stress conditions, leading to elevated Ca²⁺ and Na⁺ entering the post synaptic neuron and cell damage.

Dopaminergic neurons manufacture dopamine. Dopamine is a neurotransmitter that acts on D1 type (D1 and D5) Gs coupled receptors, which increase cAMP and PKA, and D2 type (D2, D3, and D4) receptors, which activate Gi-coupled receptors that decrease cAMP and PKA. Dopamine is connected to mood and behavior, and modulates both pre and post synaptic neurotransmission. Loss of dopamine neurons in the substantia nigra has been linked to Parkinson's disease.

Serotonergic neurons manufactures serotonin. Serotonin, (5-Hydroxytryptamine, 5-HT), can act as excitatory or inhibitory. Of the four 5-HT receptor classes, 3 are GPCR and 1 is ligand gated cation channel. Serotonin is synthesized from tryptophan by tryptophan hydroxylase, and then further by aromatic acid decarboxylase. A lack of 5-HT at postsynaptic neurons has been linked to depression. Drugs that block the presynaptic serotonin transporter are used for treatment, such as Prozac and Zoloft.

Astrocytes, also known collectively as astroglia, are characteristic star-shaped glial cells in the brain and spinal cord. They perform many functions, including biochemical support of endothelial cells that form the blood-brain barrier, provision of nutrients to the nervous tissue, maintenance of extracellular ion balance, and a role in the repair and scarring process of the brain and spinal cord following traumatic injuries.

Glial cells, sometimes called neuroglia or simply glia are non-neuronal cells that maintain homeostasis, form myelin, and provide support and protection for neurons in the brain, and for neurons in other parts of the nervous system such as in the autonomic nervous system.

Oligodendrocytes are a type of brain cell. They are a variety of neuroglia. Their main function is the insulation of axons (the long projection of nerve cells) in the central nervous system (the brain and spinal cord) of some vertebrates.

“Neuronal stem cell” or “NSC” or “neuronal precursor cell” or “NPC” refers to any cell that can differentiate in a neuronal cell.

“Parthenogentically derived neuronal stem cell” or “phNSC” refers to any cell that can differentiate in a neuronal cell that has been neuronal stem cells produced from parthenogenetically derived human stem cells.

In another embodiment, the invention provides a method for producing a neuronal stem cells by differentiating parthenogenetically derived human stem cells by a) growing parthenogenetically derived human stem cells on a feeder layer of fibroblast cells for at least 2 days; b) growing parthenogenetically derived human stem cells on a petri dish without fibroblast feeder layer for at least 1 day; c) culturing the cells in a neuronal induction media; d) obtaining a single cell suspension of the cells from (c); and e) culturing the single cells from step (d) on a petri dish with no fibroblast feeder layer in a neuronal proliferation media. In one aspect the neuronal induction media is made of Penicillin-Streptomycin-Amphotericin Solution (VWR, Radnor Pa.), DMEM/F12 (Invitrogen Grand Island, N.Y.), L-Glutamine (Invitrogen Grand Island, N.Y.), MEM Non-Essential Amino Acids Solution (Invitrogen Grand Island, N.Y.), N2 Supplement (Invitrogen Grand Island, N.Y.); and bFGF (Peprotech Rocky Hill, N.J.). In a further aspect L-Glutamine is present at 2 mM, MEM Non-Essential Amino Acids Solution is present at 0.1 mM and bFGF is present at 4-20 ng/ml in the neuronal induction media. In another aspect, the neuronal proliferation media is made of Penicillin-Streptomycin-Amphotericin Solution (VWR, Radnor Pa.), DMEM/F12 (Invitrogen Grand Island, N.Y.), GlutaMAX™-I (Invitrogen Grand Island, N.Y.), StemPro® Neural Supplement (Invitrogen Grand Island, N.Y.), 20 ng/ml bFGF (Peprotech Rocky Hill, N.J.) and 20 ng/ml EGF (Invitrogen Grand Island, N.Y.). In an additional aspect, FGF and EGF are present at 20 ng/ml in the neuronal proliferation media. In a further aspect, the petri dish is coated with CELLstart™ (Invitrogen Grand Island, N.Y.). The invention also provides for a neuronal stem cell produced by this method. In an additional aspect, a neuroepithelial rosette forms in about 1-2 weeks of culture in the neuronal induction media.

In a further embodiment, the invention provides for isolated neuronal stem cells derived from parthenogenetically derived human stem cells by a) growing parthenogenetically derived human stem cells on a feeder layer of fibroblast cells for at least 2 days; b) growing parthenogenetically derived human stem cells on a petri dish with no fibroblast feeder layer for at least 1 day; c) culturing the cells in a neuronal induction media; d) obtaining a single cell suspension of the cells from (c); and e) culturing the single cells from step (d) on a petri dish with no fibroblast feeder layer in a neuronal proliferation media. In one aspect, the neuronal stem cells express neural markers selected from the group consisting of SOX2, Nestin, Mushashi-1, TUBB3, MAP2, FOXO4, GFAP, CD113 and CD15. In another aspect, the neuronal stem cells maintain the neuronal phenotype for at least 27 passages. In another aspect the neuronal stem cells are histocompatible with the oocyte donor. In an additional aspect, the neuronal stem cell has a different pattern of zygosity from an ESC. In another aspect, the neuronal stem cell contains only the maternal genome. In a further aspect, the neuronal stem cells are transplantable to humans. In an additional aspect, the neuronal stem cells are undifferentiated, partially differentiated or fully differentiated. In a further aspect, the neuronal stem cells can be differentiated into neuronal cells. In another aspect, the neuronal cells differentiated from neuronal stem cells can be neurons, glial cells, oligodendrocytes and astrocytes. In one aspect the differentiated neuronal cell is a neuron. In a further aspect the neuron is selected from the group consisting of a cholinergic neuron, a GABAergic neuron, a glutamatergic neuron, a dopaminergic neuron and a serotonergic neuron.

Normally, the oocyte is ovulated at this stage and fertilized by the sperm. The sperm initiates the completion of meiosis in a process called activation. During activation, the pairs of chromatids separate, the second polar body is extruded, and the oocyte retains a haploid number of chromosomes, each with one chromatid. The sperm contributes the other haploid complement of chromosomes to make a full diploid cell with single chromatids. The chromosomes then progress through DNA synthesis during the first cell cycle. These cells then develop into embryos.

By contrast, embryos described herein are developed by artificial activation of cells, typically mammalian oocytes or blastomeres containing DNA of all male or female origin. As discussed in the background of the invention, many methods have been reported in the literature for artificial activation of unfertilized oocytes. Such methods include physical methods, e.g., mechanical methods such as pricking, manipulation or oocytes in culture, thermal methods such as cooling and heating, repeated electric pulses, enzymatic treatments, such as trypsin, pronase, hyaluronidase, osmotic treatments, ionic treatments such as with divalent cations and calcium ionophores, such as ionomycin and A23187, the use of anesthetics such as ether, ethanol, tetracaine, lignocaine, procaine, phenothiazine, tranquilizers such as thioridazine, trifluoperazine, fluphenazine, chlorpromazine, the use of protein synthesis inhibitors such as cycloheximide, puromycin, the use of phosphorylation inhibitors, e.g., protein kinase inhibitors such as staurosporine, 2-aminopurine, shingosine, and DMAP, combinations thereof, as well as other methods.

Such activation methods are well known in the art and are discussed U.S. Pat. No. 5,945,577, incorporated herein by reference.

In one embodiment, a human cell in metaphase II, typically an oocyte or blastomere comprising DNA of all male or female origin, is artificially activated for effecting artificial activation of oocytes.

In a related aspect, the activated cell, e.g., oocyte, which is diploid, is allowed to develop into an embryo that comprises a trophectoderm and an inner cell mass. This can be effected using known methods and culture media that facilitate blastocyst development.

After the gynogenetic embryos have been cultured to produce a discernable trophectoderm and inner cell mass, the cells of the inner cell mass are then used to produce the desired pluripotent cell lines. This can be accomplished by transferring cells derived from the inner cell mass or the entire inner cell mass onto a culture that inhibits differentiation. This can be effected by transferring the inner cell mass cells onto a feeder layer that inhibits differentiation, e.g., fibroblasts or epithelial cells, such as fibroblasts derived from postnatal human tissues, etc., or other cells that produce LIF. Other factors/components may be employed to provide appropriate culture conditions for maintaining cells in the undifferentiated state including, but not limited to, addition of conditioned media [20], bFGF and TGF-β (with or without LIF) [21], factors which activate the gp130/STAT3 pathway [22], factors which activate the PI3K/Akt, PKB pathway [23], factors that are members of the bone morphogenetic protein (BMP) super-family [22], and factors which activate the canonical/β-catenin Wnt signaling pathway (e.g., GSK-3-specific inhibitor; [24]). In a related aspect, such factors may comprise culture conditions that include feeder cells and/or ECM substrates [22].

In one aspect, the inner cell mass cells are cultured on human postnatal foreskin or dermal fibroblast cells or other cells which produce leukemia inhibitory factor, or in the presence of leukemia inhibitory factor. In a related aspect, feeder cells are inactivated prior to seeding with the ICM. For example, the feeder cells can be mitotically inactivated using an antibiotic. In a related aspect, the antibiotic can be, but is not limited to, mytomycin C.

Culturing will be effected under conditions that maintain the cells in an undifferentiated, pluripotent state, for prolonged periods, theoretically indefinitely. In one embodiment, oocytes are parthenogenically activated with calcium ionophores under high O₂ tension followed by contacting the oocytes with a serine-threonine kinase inhibitor under low O₂ tension. The resulting ICM from the parthenogenically activated oocytes are cultured under high O₂ tension, where the cells, for example, are maintained using a gas mixture comprising 20% O₂. In one aspect, culturable refers to being capable of or fit for, being cultivated. In a related aspect, ICM isolation is carried out mechanically after four days of blastocyst cultivation, where the cultivation is carried out on feeder cells. Such cultivation, for example, eliminates the need to use materials derived from animal sources, as would be the case for immunosurgery.

In a related aspect, culture media for the ICM is supplemented with non-animal sera, including but not limited to, human umbilical cord serum, where the serum is present in defined media (e.g., IVF, available from MediCult A/S, Denmark; Vitrolife, Sweden; or Zander IVF, Inc., Vero Beach, Fla.). In another aspect, the media and processes as provided are free of animal products. In a related aspect, animal products are those products, including serum, interferons, chemokines, cytokines, hormones, and growth factors, that are from non-human sources.

The pluripotent state of the cells produced by the present invention can be confirmed by various methods. For example, the cells can be tested for the presence or absence of characteristic ES cell markers. In the case of human ES cells, examples of such markers are identified supra, and include SSEA-4, SSEA-3, TRA-1-60 and TRA-1-81 and are known in the art.

Also, pluripotency can be confirmed by injecting the cells into a suitable animal, e.g., a SCID mouse, and observing the production of differentiated cells and tissues. Still another method of confirming pluripotency is using the subject pluripotent cells to generate chimeric animals and observing the contribution of the introduced cells to different cell types. Methods for producing chimeric animals are well known in the art and are described in U.S. Pat. No. 6,642,433, incorporated by reference herein.

Yet another method of confirming pluripotency is to observe ES cell differentiation into embryoid bodies and other differentiated cell types when cultured under conditions that favor differentiation (e.g., removal of fibroblast feeder layers). This method has been utilized and it has been confirmed that the subject pluripotent cells give rise to embryoid bodies and different differentiated cell types in tissue culture.

The resultant pluripotent cells and cell lines, preferably human pluripotent cells and cell lines, which are derived from DNA of entirely female original, have numerous therapeutic and diagnostic applications. Such pluripotent cells may be used for cell transplantation therapies or gene therapy (if genetically modified) in the treatment of numerous disease conditions.

In this regard, it is known that mouse embryonic stem (ES) cells are capable of differentiating into almost any cell type, e.g., neuronal stem cells. Therefore, human pluripotent (ES) cells produced according to the invention should possess similar differentiation capacity. The pluripotent cells according to the invention will be induced to differentiate to obtain the desired cell types according to known methods. For example, human ES cells produced according to the invention may be induced to differentiate into neuronal stem cells, hematopoietic stem cells, muscle cells, cardiac muscle cells, liver cells, islet cells, retinal cells, cartilage cells, epithelial cells, urinary tract cells, etc., by culturing such cells in differentiation medium and under conditions which provide for cell differentiation. Medium and methods which result in the differentiation of ES cells are known in the art as are suitable culturing conditions.

For example, Palacios et al. [25] teach the production of hematopoietic stem cells from an embryonic cell line by subjecting stem cells to an induction procedure comprising initially culturing aggregates of such cells in a suspension culture medium lacking retinoic acid followed by culturing in the same medium containing retinoic acid, followed by transferal of cell aggregates to a substrate which provides for cell attachment.

Moreover, Pedersen et al. [26] is a review article which references numerous articles disclosing methods for in vitro differentiation of embryonic stem cells to produce various differentiated cell types including hematopoietic cells, muscle, cardiac muscle, nerve cells, among others.

In a further embodiment, the invention provides a method of differentiating neuronal stem cells by culturing neuronal stem cells in neuronal differentiation media. In one aspect, the neuronal differentiation media contains Penicillin-Streptomycin-Amphotericin (VWR Radnor, Pa.); DMEM/F12 (Invitrogen Grand Island, N.Y.); GlutaMAX™-I (Invitrogen Grand Island, N.Y.); and StemPro® Neural Supplement (Invitrogen Grand Island, N.Y.). In a further aspect, the neuronal stem cells are differentiated into a neuronal cell selected from the group consisting of a neuron, a glial cell, an oligodendrocyte and an astrocyte. The invention also provides for the neuronal cells differentiated from the neuronal stem cells. In one aspect, the neuronal stem cells are produced from parthenogenetically derived human stem cells. In another aspect the neuronal stem cells are histocompatible with the oocyte donor. In an additional aspect, the neuronal stem cell has a different pattern of zygosity from an ESC. In another aspect, the neuronal stem cell contains only the maternal genome. In a further aspect, the neuronal stem cells are transplantable to humans. In an additional aspect, the neuronal stem cells are undifferentiated, partially differentiated or fully differentiated. In one aspect the differentiated neuronal cell is a neuron. In a further aspect the neuron is selected from the group consisting of a cholinergic neuron, a GABAergic neuron, a glutamatergic neuron, a dopaminergic neuron and a serotonergic neuron.

Further, Bain et al. [27] teach in vitro differentiation of embryonic stem cells to produce neural cells which possess neuronal properties. These references are exemplary of reported methods for obtaining differentiated cells from embryonic or stem cells. These references and in particular the disclosures therein relating to methods for differentiating embryonic stem cells are incorporated by reference in their entirety herein. Thus, using known methods and culture medium, one skilled in the art may culture the subject ES cells, including genetically engineered or transgenic ES cells, to obtain desired differentiated cell types, e.g., neural cells, muscle cells, hematopoietic cells, etc. Pluripotent cells produced by the methods described herein may be used to obtain any desired differentiated cell type. Therapeutic usages of differentiated human cells are unparalleled. For example, human hematopoietic stem cells may be used in medical treatments requiring bone marrow transplantation. Such procedures are used to treat many diseases, e.g., late stage cancers such as ovarian cancer and leukemia, as well as diseases that compromise the immune system, such as AIDS. Hematopoietic stem cells can be obtained, e.g., by incorporating male or female DNA derived from a male or female cancer or AIDS patient with an enucleated oocyte, obtaining pluripotent cells as described above, and culturing such cells under conditions which favor differentiation, until hematopoietic stem cells are obtained. Such hematopoietic cells may be used in the treatment of diseases including cancer and AIDS.

Alternatively, the subject pluripotent cells may be used to treat a patient with a neurological disorder by culturing such cells under differentiation conditions that produce neural cell lines. Specific diseases treatable by transplantation of such human neural cells include, by way of example, Parkinson's disease, Alzheimer's disease, ALS and cerebral palsy, among others. In the specific case of Parkinson's disease, it has been demonstrated that transplanted fetal brain neural cells make the proper connections with surrounding cells and produce dopamine. This can result in long-term reversal of Parkinson's disease symptoms.

Stem cell treatments are a type of intervention strategy that introduces new cells into damaged tissue in order to treat disease or injury. The ability of stem cells to self-renew and give rise to subsequent generations with variable degrees of differentiation capacities, offers significant potential for generation of tissues that can potentially replace diseased and damaged areas in the body, with minimal risk of rejection and side effects. Typically, stem cells are transplanted to the desired are for treatment.

In one embodiment, the invention provides a method of treating a neurologic disorder using neuronal stem cells derived from parthenogenetically derived from oocytes. A neurological disorder is a disorder of the body's nervous system. Structural, biochemical or electrical abnormalities in the brain, spinal cord or other nerves can result in a range of symptoms. Examples of symptoms include paralysis, muscle weakness, poor coordination, loss of sensation, seizures, confusion, pain and altered levels of consciousness. There are many recognized neurological disorders, some relatively common, but many rare. They may be assessed by neurological examination, and studied and treated within the specialties of neurology and clinical neuropsychology.

In one aspect, the neurologic disorder is selected from the group consisting of epilepsy, convulsions, neurotoxic injury, hypoxia, anoxia, ischemia, stroke, cerebrovascular accident, brain or spinal cord trauma, myocardial infarct, physical trauma, drowning, suffocation, perinatal asphyxia, hypoglycemic events, neurodegeneration, Alzheimer's disease, senile dementia, Amyotrophic Lateral Sclerosis, Multiple Sclerosis, Parkinson's disease, Huntington's disease, Down's Syndrome, Korsakoff's disease, schizophrenia, AIDS dementia, multi-infarct dementia, Binswanger dementia, neuronal damage, seizures, chemical toxicity, addiction, morphine tolerance, opiate tolerance, opioid tolerance, barbiturate tolerance, acute and chronic pain, migraine, anxiety, major depression, manic-depressive illness, obsessive-compulsive disorder, schizophrenia and mood disorders, bipolar disorder, unipolar depression, dysthymia, seasonal effective disorder, dystonia or other movement disorders, sleep disorder, muscle relaxation and urinary incontinence. In a further aspect, the neuronal stem cells are implanted into a patient in need of such treatment.

One object of the subject invention is that it provides an essentially limitless supply of pluripotent, human cells that can be used to produce differentiated neural cells. Human embryonic stem cells and their differentiated progeny derived from blastocysts remaining after infertility treatments, or created using NT, will likely be rejected by a recipient's immune system when used in allogenic cell transplantation therapy. Parthenogenically derived stem cells should result in differentiated cells that could alleviate the significant problem associated with current transplantation methods, i.e., rejection of the transplanted tissue which may occur because of host-vs-graft or graft-vs-host rejection relative to the oocyte donor. Conventionally, rejection is prevented or reduced by the administration of anti-rejection drugs such as cyclosporin. However, such drugs have significant adverse side-effects, e.g., immunosuppression, carcinogenic properties, as well as being very expensive. Cells produced by the methods as disclosed should eliminate, or at least greatly reduce, the need for anti-rejection drugs relative to the oocyte donor.

Another object of the subject invention is that it provides an essentially limitless supply of pluripotent, human cells that can be used to produce differentiated neuronal cells suitable for allogenic transplantation to members of the oocyte donor's family. The cells will be immunologically and genetically similar to those of the oocytes donor's direct family members and thus less likely to be rejected by the donor's family members.

For example, the gene encoding brain derived growth factor may be introduced into human pluripotent cells produced according to the invention, the cells differentiated into neural cells and the cells transplanted into a Parkinson's patient to retard the loss of neural cells during such disease.

In one embodiment, a neuronal stem cell is disclosed which is produced in vitro, in the absence of a mechanical support for control of differentiation and/or proliferation (i.e., the absence of 3-D scaffolding) In one aspect, a neuronal stem cell is disclosed, including, but not limited to, a neuronal cell that is terminally differentiated in vitro.

In another embodiment, the neuronal stem cell is produced from parthenogenetically activated human oocytes, where stem cells derivitized from the parthenogentically activated oocytes are artificially manipulated to produce the neuronal stem cell.

In one aspect, the neuronal stem cell is produced including culturing the isolated stem cells from parthenogenetically activated oocytes in media comprising serum replacement (M/SR), plasmonate, and at least one mitogen that activates the gp130/STAT pathway and/or MAP kinase pathway on a fibroblast feeder layer treated with a DNA inhibitor, culturing the mitogen treated cells in M/SR comprising plasmonate (M/SRP), without added mitogens, to near confluence, where ½ volume of the M/SRP is replaced with M/SR periodically until the near confluent cells develop pigmentation and a domed appearance, and transferring the pigmented/domed cells in M/SR to a gelatin coated substrate, where ½ volume of the M/SR is replaced with M/SR periodically until a floating cell mass develops, where the floating cell mass is the neuronal stem cells. In a related aspect, the M/SR includes KO Hi glucose DMEM, streptomycin, non-essential amino acids, Glutamax-I, β-mecaptoethanol, and Serum Replacement. In another related aspect, M/SRP comprises the components of M/SR and plasmonate.

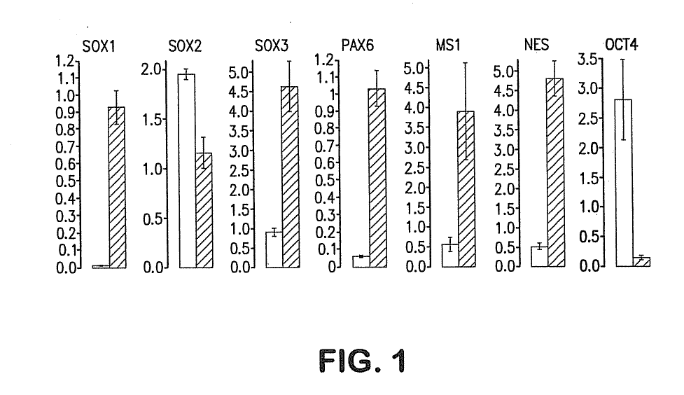

This invention is directed in one aspect toward demonstrating that NSC can be efficiently derived from hpSC. For this purpose we have chosen the adherent model [11], because it provides more uniform and synchronous formation of neuroectoderm compared with the protocol using the embryoid bodies [12]. Unlike the original protocol [11], feeder cells were not used to grow stem cell colonies for neural induction. In our study, development of NEP rosettes in hpSC colonies grown on CELLstart occurred within a week after replacement of ES-medium with medium for neural induction. NEP rosettes obtained from hpSC had well formed lumen and expressed appropriate neural marker set, which provides evidence for the adequate formation of neuroepithelium. It is noteworthy that increased expression of SOX1 and SOX3 was observed in the hpSC-derived NEP rosettes, whereas a slight decrease of SOX2 expression was found (FIG. 1), that might be associated with OCT4 down-regulation [13].

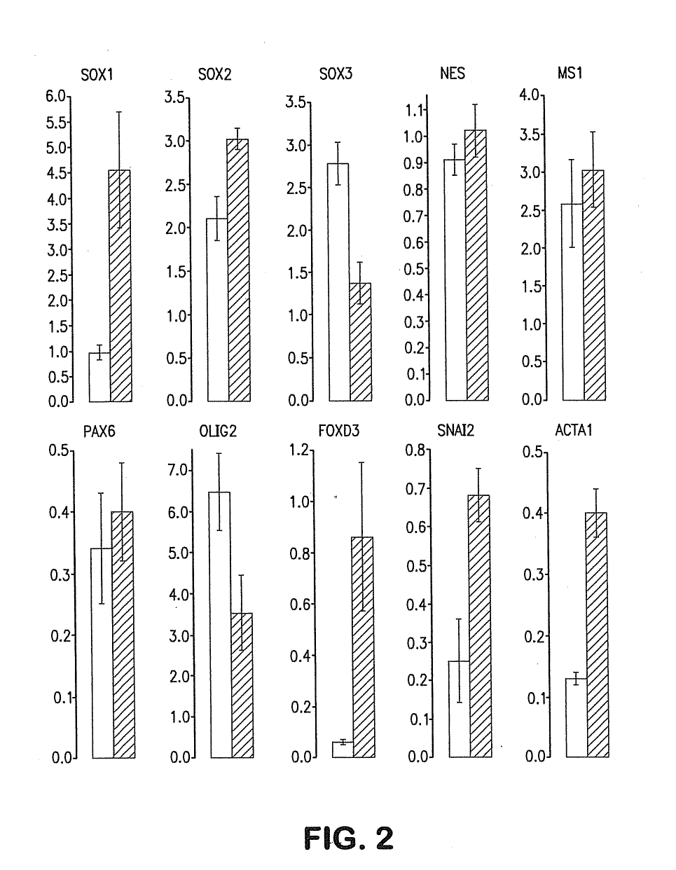

The properties of phNSC were compared with hNSC, which was derived from hESC H9. Transcriptional activity of main genes specific for NSC was comparable for both cell lines. Despite this, the expression of SOXB1 genes was different in phNSC and hNSC (FIG. 2). The exact roles of SOXB1 genes in maintenance of neural progenitors and in restriction of their differentiating abilities remain still unclear. It was shown that the functions of these genes are redundant [13, 14], thus it is possible that in maintaining the properties of NSC, SOXB1 genes are mutually compensate each other.

Most phNSC obtained expressed surface markers of neuroectoderm CD113 and CD15, but this expression wasn't uniform. Sun et al. [15] showed that undifferentiated human and murine NSC could represent heterogeneous CD113 and CD15 populations, and the expression of markers depends on the phase of the cell cycle. Despite this, CD113 negative cells are capable of maintaining their proliferative and neurogenic potential [15].

To support NSC proliferation growth factor bFGF is needed, but this inhibits endogenous SHH, leading to a rapid loss of ability to differentiate into neurons and promotes metamorphosis of NSC into neural crest ectomesenchymal cells [16]. The expression levels of neural crest marker genes FOXD3 and SNAI2, and mesodermal marker ACTA1 were not high in phNSC up to 27 passages, and even lower in comparison with hNSC (FIG. 2). These data indicate the absence of large-scale metamorphosis of hNSC into ectomesenchymal cells.

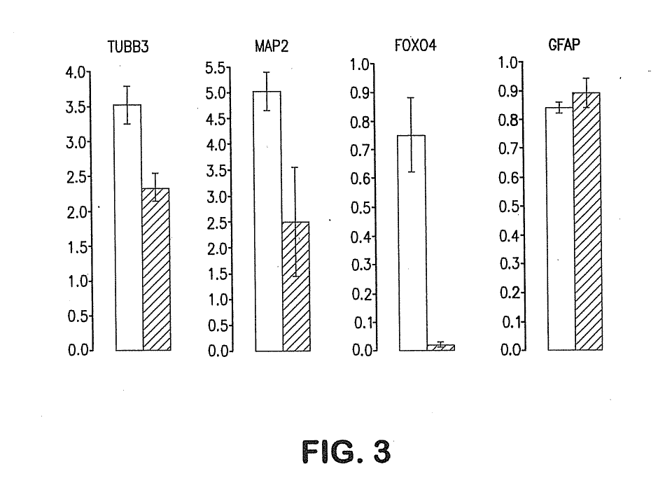

Resulting from spontaneous differentiation in the medium without growth factors, a significant part of the cell population was represented by neurons in the case of phNSC, as well as in the case of hNSC. Neuronal differentiation was confirmed by positive immunocytochemical staining for Tuj 1 (tubulin βIII) and by high transcriptional activity of TUBB3 and MAP2 genes, revealed by qRT-PCR analysis (FIG. 3). Transcriptional activity of specific oligodendrocyte marker FOXO4 and astrocyte marker GFAP indicated the presence of glial derivatives among differentiated cells. We therefore conclude that the phNSC obtained can be considered as precursors of all three main types of neural derivatives.

Thus, this invention has demonstrated that pluripotent hpSC can serve as a good source of NSC. phNSC obtained are capable of relatively long-term proliferation while maintaining their neurogenetic potential and ability to provide sufficient quantity of cells for cryopreservation and further implementation.

Multipotent neural precursor cells (NPC) have been derived from neuroectoderm which was derived from parthenogenetic stem cells either homozygous or heterozygous. The parthenogenetically derived NPCs differentiate into neurons such as midbrain dopaminergic neurons (DA). These DA neurons exhibit a midbrain phenotype and express TH, GIRK2, PITX3, NURR1, LMXA1, and EN1 as measured by immunocytochemistry and RT-PCR. As it is known from prior art, the main function of dopaminergic neurons is to release dopamine. Dopamine's major function in the body is reward-driven learning. The DA neurons derived from hpNPC also release dopamine as determined by LC/MS/MS. Whole cell electrophysiology proved that the parthenogenetically derived dopaminergic neurons are capable of firing action potentials.

In one embodiment, the invention provides for a method for producing neuronal stem cells by differentiating parthenogenetically derived human stem cells by: a) cultivation of human pluripotent stem cells in feeder-free conditions; b) exposure of said cells to neuronal induction medium; c) mechanical isolation of partially differentiated cells; and d) further expansion and maintenance of said cells until maturation. In one aspect the neuronal induction media comprises: a) Penicillin-Streptomycin-Amphotericin Solution; b) DMEM/F12; c) MEM Non-Essential Amino Acids Solution; d) L-Glutamine; e) N2 Supplement; and f) bFGF. In a further aspect L-Glutamine is present at 2 mM, MEM Non-Essential Amino Acids Solution is present at 0.1 mM and bFGF is present at 4-20 ng/ml in the neuronal induction media. In another aspect the neuronal proliferation media comprises: a) Penicillin-Streptomycin-Amphotericin; b) DMEM/F12; c) GlutaMAX™-I; d) StemPro® Neural Supplement; e) bFGF; and f) EGF. In an additional aspect, FGF and EGF are present at 20 ng/ml in the neuronal proliferation media. In an additional aspect the feeder-free conditions utilize the ECM substrate including but not limited to: CELLstart, Matrigel, laminin, gelatin, fibronectin. The invention also provides for the neuronal stem cell produced by the method. In a further aspect, a neuroepithelial rosette forms after 1-2 weeks.

In an additional embodiment, the invention provides for isolated neuronal stem cells derived from parthenogenetically derived human stem cells using the method comprising: a) cultivation of human pluripotent stem cells in feeder-free conditions; b) exposure of said cells to neuronal induction medium; c) mechanical isolation of partially differentiated cells; and d) further expansion and maintenance of said cells until maturation. In one aspect, the cells express neural stem cell markers selected from the group consisting of: SOXB1-family NES, MSH-1, CXCR4, CCND1, LHX2, PAX6, GAP43. In a further aspect the neuronal stem cells maintain the neuronal phenotype for at least 30 passages. In one aspect, the neuronal stem cells can differentiate into neuronal cells. In an additional aspect, the neuronal cells are selected from the group consisting of neurons, astrocytes and oligodendrocytes. In one aspect the differentiated neuronal cell is a neuron. In a further aspect the neuron is selected from the group consisting of a cholinergic neuron, a GABAergic neuron, a glutamatergic neuron, a dopaminergic neuron and a serotonergic neuron.

In a further embodiment, the invention provides a method of treating a neurologic disorder using neuronal stem cells derived from parthenogenetically derived from oocytes. In one aspect the neurologic disorder is selected from the group consisting of: epilepsy, convulsions, neurotoxic injury, ischemia, stroke, cerebrovascular accident, brain or spinal cord trauma, physical trauma, Alzheimer's disease, senile dementia, Amyotrophic Lateral Sclerosis, Multiple Sclerosis, Parkinson's disease, Huntington's disease, schizophrenia, neuronal damage, migraine, anxiety, major depression, manic-depressive illness, obsessive-compulsive disorder, schizophrenia and mood disorders, bipolar disorder, unipolar depression, dystonia or other movement disorders, sleep disorder, muscle relaxation. In a further aspect, the neuronal stem cells are implanted into a patient in need of such treatment.

In one embodiment, the invention provides for a method of differentiating neuronal stem cells, the method comprising culturing neuronal stem cells in neuronal differentiation media. In one aspect the neuronal differentiation media comprises: a) Penicillin-Streptomycin-Amphotericin; b) DMEM/F12; c) GlutaMAX™-I; and d) StemPro® Neural Supplement. In an additional aspect, the neuronal stem cells are differentiated into a neuronal cell selected from the group consisting of: a neuron, an oligodendrocyte and an astrocyte. In one aspect the differentiated neuronal cell is a neuron. In a further aspect the neuron is selected from the group consisting of a cholinergic neuron, a GABAergic neuron, a glutamatergic neuron, a dopaminergic neuron and a serotonergic neuron. The invention also provides for the differentiated cells produced by this method. In one aspect, the cells are differentiated from a parthenogenetically activated oocyte.

The following examples are intended to illustrate but not limit the invention.

### Example 1

### Production of Human Parthenogenic Embryogenic Stem Cells

Materials and Methods

Donors voluntarily donated eggs and blood (for DNA analysis) with no financial payment. Donors signed comprehensive informed consent documents and were informed that all donated materials were to be used for research and not for reproductive purposes. Before ovarian stimulation, oocyte donors underwent medical examination for suitability according to FDA eligibility determination guidelines for donors of human cells, tissues, and cellular and tissue-based products (Food and Drug Administration. (Draft) Guidance for Industry: Eligibility Determination for Donors of Human Cells, Tissues, and Cellular and Tissue Based Products (HCT/Ps) dated May 2004) and order N 67 (02.26.03) of Russian Public Health Ministry. It included X-ray, blood and urine analysis, and liver function test. Donors were also screened for syphilis, HIV, HBV, and HCV.

Oocytes were obtained using standard hormonal stimulation to produce superovulation in the subject donor. Each donor egg underwent ovarian stimulation by FSH from the 3rd to the 13th days of their menstrual cycle. A total of 1500 IU of FSh was given. From the 10th to the 14th day of the donor's menstrual cycle, gonadoliberin antagonist Orgalutran (Organon, Holland) was injected at 0.25 mg/day. From the 12th to the 14th day of the donor's menstrual cycle a daily injection of 75 IU FSH+75 IU LH (Menopur, Ferring GmbH, Germany (was given, If an ultrasound examination displayed follicles between 18 and 20 mm in diameter, a single 8000 IU dose of hGC (Choragon, Ferring GmbH, Germany) was administered on the 14th day of the donor's menstrual cycle. Trans-vaginal punction was performed 35 hours after hCG injection on approximately the 16th day. Follicular fluid was collected from the antral follicles of anesthetized donors by ultrasound-guided needle aspiration into sterile tubes.

Cumulus oocyte complexes (COCs) were picked from the follicular fluid, washed in Flushing Medium (MediCult) and then incubated in Universal IVF medium (MediCult, see Table 1) with a Liquid Paraffin (MediCult) overlay for 2 hours in a 20% O₂, 5% CO₂, at 37° C. humidified atmosphere.

TABLE 1
IVF media.
COMPOSITION

Calcium Chloride
EDTA
Glucose
Human Serum Albumin
Magnesium Sulfate
Penicillin G
Potassium Chloride
Potassium di-Hydrogen Phosphate
Sodium Bicarbonate
Sodium Chloride
Sodium Lactate
Sodium Pyruvate
Water

Before activation, cumulus-oocyte complexes (COCs) were treated with SynVitro Hyadase (MediCult) to remove cumulus cells followed by incubation in Universal IVF medium with a paraffin overlay for 30 minutes.

From this point onward, the culture of oocytes and embryos was performed in a humidified atmosphere at 37° C. using O₂-reduced gas mixture (90% N₂+5% O₂+5% CO₂), with the exception of the ionomycin treatment. The oocytes were activated by incubation in 5 ionomycin for 5 minutes in a CO₂ incubator at 37° C. in a gas environment of 20% O₂, 5% CO₂, followed by culture with 1 mM 6-dimethylaminopurine (DMAP) for 4 hours in IVF medium, paraffin overlay, in a gas environment of 90% N₂, 5% O₂, and 5% CO₂ at 37° C. Activation and cultivation were carried out in 4-well plates (Nunclon, A/S, Denmark) in 500 μl of medium overlaid with liquid paraffin oil (MediCult, A/S, Denmark).

Activated oocytes were cultivated in IVF medium in a gas environment comprising 5% O₂, 5% CO₂, and 90% N₂, and embryos generated from the activated oocytes were cultured in the same gas mixture.

Activated oocytes were allowed to incubate in IVF under the above conditions until fully expanded blastocysts containing an inner cell mass (ICM) at a Blastocyst Scoring Modification of 1 AA or 2AA (Shady Grove Fertility Center, Rockville, Md., and Georgia Reproductive Specialists, Atlanta, Ga.) was observed.

The zona pellucida was removed by 0.5% pronase (Sigma, St. Louis) treatment. The ICM from blastocysts was isolated by immuno-surgery where the blastocysts were incubated with horse antiserum to human spleen cells followed by exposure to guinea pig complement. Trophoectoderm cells were removed from the ICM by gently pipetting the treated blastocysts.

For the derivation of phESC from whole blastocysts, the blastocysts were placed on a feeder layer in medium designed for culture of phESC (i.e., VitroHES (Vitrolife) supplemented with 4 ng/ml hrbFGF, 5 ng/ml hrLIF and 10% human umbilical cord blood serum). When blastocysts attached and trophoplast cells spread, the ICM became visible. Through three to four days of additional culture, the ICM was isolated through mechanical slicing of the ICM from the trophoectoderm outgrowth using a finely drawn glass pipette. Further, the IMC cells were cultured on a feeder cell layer of mitotically inactivated post natal human dermal fibroblasts, in VITROHES™ media (e.g., DMEM/high glucose medium, VitroLife, Sweden) supplemented with 10% human umbilical cord blood serum, 5 ng/ml human recombinant LIF (Chemicon Inc., Temecula, Calif.), 4 ng/ml recombinant human FGF (Chemicon Int'l, Inc., Temecula, Calif.) and penicillin-streptomycin (100 U/100 μg) in a 96-well plate in 5% CO₂ and 20% O₂ at 37° C. This gas mixture was used to culture stem cells. Human fibroblast cultures were made using non-animal materials. Inactivation of fibroblasts was carried out using 10 μg/ml mitomycin C (Sigma, St. Louis, Mo.) for 3 hours.

In a separate method, immuno-surgery was performed by incubating blastocysts with horse antiserum to human spleen cells followed by exposure to rabbit complement. The trophectoderm cells were removed from the ICM through gentle pipetting of the treated blastocyts. Further culturing of the isolated ICMs was performed on a feeder layer of neonatal human skin fibroblasts (HSF) obtained from a genetically unrelated individual (with parental consent) derived using medium containing human umbilical cord blood serum. The HSF feeder layer was mitotically inactivated using mitomycin C.

The medium for the culture of HSF consisted of 90% DMEM (high glucose, with L-glutamaine (Invitrogen), 10% human umbilical cord blood serum and penicillin-streptomycin (100 U/100 mg) Invitrogen).

For the culture of ICM and phESC, VitroHES (Vitrolife) supplemented with 4 ng/ml hrbFGF, 5 ng/ml hrLIF and 10% human umbilical cord blood serum was used. The ICM was mechanically plated on a fresh feeder layer and cultured for three to four days. The first colony was mechanically cut and replated after five days of culture. All subsequent passages were made after five to six days in culture. For early passages, colonies were mechanically divided into clumps and replated. Further passing of phESC was performed with collagenase IV treatment and mechanical dissociation. The propagation of phESC was performed at 37° C., 5% CO₂ in a humidified atmosphere.

Oocyte Activation

From the initial 4 donors, activated oocytes were cultivated in IVF medium in a gas environment comprising 5% O₂, 5% CO₂, and 90% N₂ and followed over five (5) days. Table 2 shows the progress of maturation of the activated oocytes. Each oocyte was separated in a 4-well plate.

TABLE 2
Cultured Activated Oocytes.*

| | Day 1 | Day 2 | Day 3 | Day 5 |
|---|---|---|---|---|
| N1 | 1 pronucleus (pn), | 2 blastomers | 4 bl equal, | 1 morula, |
| | 1 polar body (pb) | (bl) equal, | fr-2% | fr-15% |
| | | fragmentation | | |
| | | (fr)-0% | | |
| N2 | 0 pn, | 4 bl not equal, | 5 bl not equal, | 4 bl not equal, |
| | 1 pb | fr-4% | fr-20% | fr-40% |
| N3 | 1 pn, | 2 bl not equal, | 6 bl equal, | early |
| | 1 pb | fr-0% | fr-0% | blastocysts |
| N4 | 1 pn, | 4 bl equal, | 4 bl equal, | Fully expanded |
| | 1 pb | fr-10% | fr-20% | blastocyst with |
| | | | | good ICM 1AA |

*Cells were incubated in M1 media (MediCult) on the first day and M2 media (Medicult) on days 2-5. Media was changed everyday. M1 and M2 contain human serum albumin, glucose and derived metabolites, physiological salts, essential amino acids, non-essential amino acids, vitamins, nucleotides, sodium bicarbonate, streptomycin (40 mg/l), penicillin (40.000 IU/l) and phenol red.

Inner cell masses were isolated from N4 and transferred to human fibroblast feeder cells as outlined above. N1 and N2 degenerated on Day 6. Further, on Day 6, N3 produced fully expanded blastocyst with ICM 2AB. N3 was then transferred to human fibroblast feeder cells on Day 6. ICM from N4 was unchanged. N3 was used to isolate stem cells.

ICM cells were cultivated in NitroHES medium in a gas environment comprising 5% CO₂ and 95% N₂ and followed over forty-five (45) days. Table 2a shows the progress of N3 ICM cell cultivation.

TABLE 2a
Progress of N3-ICM Cultivation.*

| | |
|---|---|
| Day 3 | ICM transplanted on fresh feeder cells. |
| Day 8 | Colony of cells divided mechanically into 6 pieces and |
| | cultivated in 3 wells of a 96-well plate-1st passage. |
| Day 14 | From five (5) colonies of 1st passage, cells were |
| | mechanically divided, and 20 colonies of a 2nd passage |
| | were cultivated in 3 wells of a 24-well plate. |
| Day 20 | Cells were plated in 35 mm dish-3rd passage. |
| Day 24 | Five (5) 35 mm dishes were seeded with cells-4th passage. |
| | One dish was divided chemically with 5% pronase (Sigma) |
| | at room temperature. |
| Day 30 | Twenty-five (25) 35 mm were seeded with cells-5th** |
| | passage. |
| Day 34 | 6th** cell passage. |
| Day 35 | 11 ampules were frozen from the 6th passage. |
| Day 37 | 7th** cell passage. |
| Day 44 | 12 ampules were frozen from the 7th passage. |
| Day 45 | 8th cell passage. |

*Cells were grown on M2 media (MediaCult).
**These passages were made with pronase digestion.

Stem Cell Isolation.

From the oocyte from 5 donors, the use of MediCult media is followed by a culture under reduced oxygen allowed for the production of 23 blastocysts on the fifth or sixth day of culture. Eleven of the blastocysts had visible ICMs (Table 3).

TABLE 3
Generation of parthenotes and parthenogenetic embryonic stem cell lines.

| | | | | | Blastocysts derived | | |
|---|---|---|---|---|---|---|---|
| | | | Normally | | | Without | |
| Donor | Oocytes | Oocytes | activated | Parthenotes | With | visible | Lines |
| Number | harvested | donated | oocytes | created | ICM | ICM | generated |
| 1 | 8 | 4 | 4 | 4 | 2 | — | phESC-1 |
| | | | | | | | immunosurgery |
| 2 | 15 | 8 | 8 | 8 | 3 | 3 | phESC-3 |
| | | | | | | | phESC-4 |
| | | | | | | | phESC-5 |
| | | | | | | | all from whole |
| | | | | | | | blastocysts |
| 3 | 27 | 14 | 12¹ | 11² | 3 | 2 | phESC-6 |
| | | | | | | | from whole |
| | | | | | | | blastocysts |
| 4 | 22 | 11 | 10³ | 10 | 2 | 3 | phESC-7 |
| | | | | | | | from whole |
| | | | | | | | blastocysts |
| 5 | 20 | 9⁴ | 7 | 7 | 1 | 4 | No cell line |
| | | | | | | | generated |

¹two oocytes were not activated;
²one oocyte degenerated after activation;
³one oocyte was not activated;
⁴two oocytes were at metaphase stage I and were discarded.

These results indicate an approximate 57.5% success rate in the formation of blastocysts from parthenogenetically activated oocytes.

### Example 2

### Maintenance of Human Parthenogenetic Stem Cells

Human parthenogenetic stem cell lines, produced in a similar manner as described above, phESC-1, phESC-3 [1] and hpSC-Hhom-4 [2], were maintained on Mitomycin-C inactivated mouse embryonic fibroblasts (Millipore) feeder layer in ES-medium: KDMEM/F12 (Invitrogen), supplemented with 15% KSR (Invitrogen Grand Island, N.Y.), 2 mM L-glutamine (GlutaMAX-I, Invitrogen Grand Island, N.Y.), 0.1 mM MEM nonessential amino acids (Invitrogen), 0.1 mM β-mercaptoethanol (Invitrogen Grand Island, N.Y.), penicillin/streptomycin/amphotericin B (100 U/100 μg/250 ng) (MP Biomedicals) and 5 ng/ml bFGF (Peprotech). Cells were passaged with Dispase or Collagenase IV (both Invitrogen Grand Island, N.Y.) every 5-7 days with split ratio of 1:4 or 1:6. There were no obvious differences in experimental results from the hpSC lines used in our study, so the data were pooled.

Good results in obtaining of Neuroepithelial Rosettes can be achieved with maintaining of hESCs on feeder layers. One passage prior to neural induction hESCs are passed on CELLstart™ coated vessels. Best results have been achieved using 60 mm Petri dishes. The day of passaging is considered as “Day 0”. hESCs are maintained during 4-7 days in the media for ES cells with 15% KSR and 5 ng/ml of bFGF. Colonies should be well formed.

### Example 3

### Materials and Methods for Culture Media Preparation and Petri Dish Coating

Materials

Knockout DMEM/F12, Invitrogen, 12660-012

DMEM/F12, Invitrogen, 10565-018, (supplemented with GlutaMAX™-I Supplement as a source of L-Glutamine).

### GlutaMAX™-I Supplement, Invitrogen, 35050-061

### MEM Non-Essential Amino Acids Solution 10 mM (100×), Invitrogen, 11140-050 CELLstart™, Invitrogen, A10142-01

### StemPro® Accutase® Cell Dissociation Reagent, Invitrogen, A11105-01 StemPro Neural Supplement, Invitrogen, A10508-01

### N2 Supplement (100×), Invitrogen, 17502-048

Dulbecco's Phosphate-Buffered Saline (D-PBS) (1×), Invitrogen, 14040-133, (with Ca2+ and Mg2+)

### EGF Recombinant Human, Invitrogen, PHG0314

### Recombinant Human FGF-basic, Peprotech, 100-18B

### Penicillin-Streptomycin-Amphotericin Solution (100×), VWR, 1674049

### Dulbecco's Phosphate-Buffered Saline (D-PBS) (1×), w/o Ca2+, Mg2+, VWR, 16777-150

Medium for Neural Induction

The title and composition of the medium are described in Shin et al. [11]. Medium for neural induction DN2 based on DMEM/F12 supplemented with N2.

Add 5 ml of 100× Penicillin-Streptomycin-Amphotericin Solution to new bottle of medium DMEM/F12 with volume of 500 ml. Store at +4. To prepare DN2 medium:
Transfer aseptically 98 ml of DMEM/F12 containing PSA solution to a sterile media bottle;
Add 1 ml of 100×MEM Non-Essential Amino Acids Solution;
Add 1 ml of 100×N2 Supplement;
Medium DMEM/F12 already contains L-Glutamine, so GlutaMAX™-I Supplement shouldn't be added
Store at +4. Add bFGF solution before use to the final concentration of 4-20 ng/ml.

Medium for Neural Proliferation

Prepare medium for neural proliferation as indicated in the manual to StemPro® NSC SFM Kit. Also StemPro® NSC SFM medium can be prepared from separate components.

Add 5 ml of 100× Penicillin-Streptomycin-Amphotericin Solution to new bottle of medium DMEM/F12 with volume of 500 ml. Store at +4. To prepare StemPro® NSC SFM medium:
Transfer aseptically 97 ml of DMEM/F12 containing PSA solution to a sterile media bottle;
Add 1 ml of 100× GlutaMAX™-I Supplement (1.1.3.);
Add 2 ml of StemPro® Neural Supplement (1.1.7.);
Store at +4. Add growth factors bFGF and EGF before use to the final concentration of 20 ng/ml.

Note: MEM Non-Essential Amino Acids Solution shouldn't be added following Invitrogen instructions.

Coating of Cultural Vessels with CELLstart™ Matrix

Dilute CELLstart™ solution in PBS with dilution factor of 50, i.e. 20 ul of CELLstart™ solution per each 1 ml of PBS. Presence of Ca2+ and Mg2+ is essential! Do not store the solution, prepare immediately before use.

Add 0.7-1.0 ml of solution per one 35 mm Petri Dish or 2.0 ml of solution per one 60 mm Petri Dish. Place in incubator at +37° C. for 2 hours. Incubation less than 2 hours or longer than 4 hours, using of dilution factor equal 100, or storage at +4° C. results in decrease of cell adhesion after passaging or during long-term cultivation.

Aspirate CELLstart™ solution before use. Do not rinse. Add culture medium immediately.

Growth Factors Solutions

Dilute growth factors EGF and bFGF in 0.1% HSA solution in PBS to the concentration of 10 ug/ml. For instance, aseptically dilute 50 ug of lyophilized growth factor in 5 ml of 0.1% HSA solution in PBS. Aliquote in 500 ul microcentrifuge tubes and store at −20° C. Avoid repetitive freeze-thaw cycles, use no longer than 14 days after thawing. Add growth factors to the media immediately before use. For instance, add 2 ul of growth factor solution per each 1 ml of media to receive final concentration of 20 ng/ml.

### Example 4

### Analysis of hpESC, phNSC and hNSC

Total RNA was isolated using the QIAsymphony automatic purification system, according to the manufacturer's instructions (Qiagen). 100-500 ng total RNA was used for reverse transcription with the iScript cDNA synthesis kit (Biorad). To analyze transcriptional activity of genes PCR reactions were performed in duplicate using 1/25-th of the cDNA per reaction and the QuantiTect Primer Assay (primers used are reported in Table 4) together with Quantitest SYBR Green master mix (Qiagen). Reverse transcriptase real-time quantitative PCR (qRT-PCR) was performed using the Rotor-Gene Q (Qiagen). Relative quantification was performed against a standard curve and quantified values were normalized against the input determined by PPIG (Cyclophilin G). After normalization, the standard error of mean of the 2-7 gene expression measurements was calculated.

FACS analysis of surface markers was performed with APC-stained mouse anti-human CD133 antibodies (eBioscience) and mouse anti-human CD15 antibodies (BD Pharmingen) (see Table 5).

For immunostaining, NSC were fixed with 4% paraformaldehyde, permeabilized by a solution containing 0.1% Tween20, and by 0.3% Triton X-100, for 1 hour after fixation. After permeabilization, the cells were blocked with 3% normal goat serum, at +4° C., overnight. The primary antibodies against SOX2, Nestin and Musashi-1 were applied overnight at +4° C. in the dilutions: 1:100, 1:200 and 1:300 respectively. The secondary antibodies (1:500) were applied for 2 hours, on the room temperature. For one-step staining of differentiated neurons, anti-Tubulin βIII Alexa Fluor 488 coupled antibodies were applied according to the manufacturer's instruction (Covance). The nuclei were stained with DAPI. The list of primary and secondary antibodies is given in Table 5.

TABLE 4
Real-time PCR primers.

| Gene | Catalog # | Producer |
|---|---|---|
| ACTA1 | QT00199815 QuantiTect Primer Assay | Qiagen |
| AFP (α-fetoprotein) | QT00085183 QuantiTect Primer Assay | Qiagen |
| FOXD3 | QT01018794 QuantiTect Primer Assay | Qiagen |
| FOXO4 | QT00029141 QuantiTect Primer Assay | Qiagen |
| GFAP | QT00081151 QuantiTect Primer Assay | Qiagen |
| MAP2 | QT00057358 QuantiTect Primer Assay | Qiagen |
| MS1 (Musashi-1) | QT00025389 QuantiTect Primer Assay | Qiagen |
| NES (Nestin) | QT00235781 QuantiTect Primer Assay | Qiagen |
| OLIG2 | QT01156526 QuantiTect Primer Assay | Qiagen |
| PAX6 | QT00071169 QuantiTect Primer Assay | Qiagen |
| POU5F1 (OCT4) | QT00210840 QuantiTect Primer Assay | Qiagen |
| SNAI2 (Slug) | QT00044128 QuantiTect Primer Assay | Qiagen |
| SOX1 | QT01008714 QuantiTect Primer Assay | Qiagen |
| SOX2 | QT00237601 QuantiTect Primer Assay | Qiagen |
| SOX3 | QT00212212 QuantiTect Primer Assay | Qiagen |
| TUBB3 | QT00083713 QuantiTect Primer Assay | Qiagen |
| PPIG (Cyclophilin | QT01676927 QuantiTect Primer Assay | Qiagen |
| G) | | |

TABLE 5
Antibodies for immunostaining and FACS.

| Antigen | Catalog # | Producer |
|---|---|---|
| CD133 | 17-1338 | eBioscience |
| Isotype control | 17-4714 | eBioscience |
| CD15 | 551376 | BD Pharmingen |
| Isotype control | 555585 | BD Pharmingen |
| Tubulin βIII | A488-435L | Covance |
| Sox-2 | ab92494 | AbCam |
| Musashi 1 | ab52865 | AbCam |
| Nestin | MAB5326 | Millipore |
| Goat anti-mouse, −488 | 35503 | ThermoScientific |
| Goat anti-rabbit, −488 | 35553 | ThermoScientific |
| Goat anti-mouse, −549 | 35508 | ThermoScientific |
| Goat anti-rabbit, −549 | 35558 | ThermoScientific |

### Example 5

### Neural Induction and Neural Stem Cells

Adherent Model has been proposed by Shin et al. [11]. Human Embryonic Stem Cells are maintained on feeder layer or matrix before they get ready to be passaged. At this time media is replaced with one for neural induction. In such conditions after 1-2 weeks of maintenance rosettes of neuroepithelial cells are formed, they are considered to be recapitulation of neural tube. Protocol of obtaining of Neuroepithelial Rosettes in feeder-free conditions on CELLstart™ is described below.

When the hESCs culture is ready to be passaged, replace culture medium with DN2 medium supplemented with 20 ng/ml of bFGF. Day of media replacement is considered “Day NI”. Media should be replaced with fresh one at least once every other day or more frequently.

After 3-4 days the rate of cell death increases significantly, the color of the media will change from red-orange to yellow fast. During this period the media should be replaced at least once a day.

After stabilization of cell death approximately in 7-10 days fields can be found, where cells form dense <<hills>> or <<ridges>> rather than monolayer. In these <<hills>> early neuroepithelial rosettes are formed.

After rosettes have begun to form, the culture should be cultivated during additional 3-7 days, until well seen areas with multiple NEP rosettes with small lumen in the center will be formed. These are late rosettes or definitive neuroectoderm. Areas, containing rosette structures, can form branched crests with long lumen, surrounded by columnar neuroepithelial cells.

For neural differentiation an adherent model [11] was used with unique and important modifications. hpSC maintained on the mouse embryonic fibroblasts feeder layer for a 5 days were passaged with Dispase (Invitrogen Grand Island, N.Y.) on CELLstart (Invitrogen Grand Island, N.Y.) coated 60 mm Petri dishes. During next 4 days colonies of hpSC were cultivated in ES-medium, followed by replacing with the medium for neural induction. Medium for neural induction is based on DMEM/F12 containing N2 supplement (Invitrogen Grand Island, N.Y.), 0.1 mM MEM nonessential amino acids, 2 mM L-glutamine (GlutaMAX-I, Invitrogen Grand Island, N.Y.), antibiotic solution and 20 ng/ml of bFGF. The day of medium replacement was considered as Day 0 of neural induction. The areas with well-formed rosettes of neuroepithelial cells were isolated mechanically, dissociated to the single cell suspension using TrypLE (Invitrogen Grand Island, N.Y.) and transferred into the CELLstart™ (Invitrogen Grand Island, N.Y.) coated wells of 24 well plate, in the StemPro NSC SFM medium (Invitrogen Grand Island, N.Y.), supplemented with 20 ng/ml of bFGF and 20 ng/ml of EGF (both Peprotech). After obtaining of sufficient amount of cells further maintaining and passaging of NSC was performed on CELLstart coated 60 mm Petri dishes, in the StemPro NSC SFM medium supplemented as described above. Cells have been dissociated by Accutase (Invitrogen Grand Island, N.Y.) during passaging. H9 hESC-Derived GIBCO® Human Neural Stem Cells (further called as hNSC; Invitrogen Grand Island, N.Y.) were maintained under the same conditions.

After 5 days of growing on CELLstart, colonies of hpSC looked fully formed and did not have any visual differences in comparison with colonies grown on feeders. After the medium was replaced, first signs of neuroepithelial (NEP) rosettes appear on the 2^{nd} day of neural induction. On the 7^{th} day of cultivation in medium for neural induction cell colonies appeared as large areas containing clusters of NEP rosettes. In these clusters most rosettes had well-formed lumen. qRT-PCR analysis revealed that transcriptional activity of the key neuroectodermal genes PAX6 and SOX1 were increased at this stage in comparison with undifferentiated hpSC, whereas pluripotency marker OCT4 was dramatically down-regulated (FIG. 1). The expression of specific neural markers NES (Nestin) and MS1 (Musashi-1) was also high. Endodermal marker AFP and mesodermermal marker ACTA1 were not detected by qRT-PCR in the NEP rosettes containing cell clusters (data not shown).

To prepare 35 mm Petri dishes, first treat them with CELLstart™ as described above, then add 2 ml of StemPro® NSC SFM medium for neural proliferation supplemented with 20 ng/ml of both bFGF and EGF. Place dishes in the incubator at +37° C., 5% CO₂, humidified atmosphere.

Late rosettes NEP, obtained during neural induction (approximately 21^{St} day after inoculation on CELLstart™ treated vessels) with lumen are isolated mechanically under stereomicroscope. One can use syringe needles. Areas with rosettes should be cut for it's impossible to isolate mechanically single cells. Areas without rosettes, as well as monolayer fields should be discarded. Collect obtained cell clumps in the minimal volume of the medium. Inoculate 15-20 clumps with size from 100 up to 300 um per one 35 mm Petri dish.

Clumps of cells then should be triturated to single cell suspension. For this purpose place 300 ul of StemPro® Accutase® in the 15 ml centrifuge tube with conical bottom and warm to 37° C. Transfer obtained clumps of cells in the minimal volume of medium in the tube with StemPro® Accutase®, incubate at room temperature for 3-4 minutes and pipet gently for approximately 100 times up and down with 200 ul tip. The total time in StemPro® Accutase® for cells shouldn't exceed 10 minutes.

Then add 6 ml of warm StemPro® NSC SFM medium for neural proliferation without growth factors. Close the lid and shake the tube gently 6-8 times to rinse the cells.

Centrifuge at 120-130 g for 4 minutes, then carefully aspirate as much supernatant as possible. Transfer 2 ml of medium from the prepared dish in the centrifuge tube, resuspend the pellet and transfer the contents of the tube in the culture vessel. Distribute cells equally in the dish by shaking it, place the dish in the incubator at +37° C., 5% CO₂, humidified atmosphere.’

Estimate cell adhesion and viability on the next day after isolation, replace the medium with the fresh one. Cells received can be considered as NSCs of the 0 passage.

### Example 6

### Clonal Isolation of phNSC

Generally populations of proliferating cells isolated from NEP rosettes aren't homogeneous. They can be contaminated with cells of mesenchymal type, which induce differentiation of NSCs and substitute them because of high proliferation rate. Isolation of individual cell clones allows obtain homogeneous populations of NSCs. To prepare culture dishes 35 mm treat them with CELLstart™, than add 2 ml of medium for neural induction DN2 supplemented with 20 ng/ml of bFGF on each dish, place the dishes in incubator at 37°, 5% CO₂, humidified atmosphere. Late NEP rosettes (approximately 21 days after inoculation of hESCs over CELLstart™) with lumen isolate mechanically under stereomicroscope as was described above. The size of the fragments with NEP rosettes should vary from 100 up to 300 μm. Inoculate 15-20 fragments with NEP rosettes in each culture dish 35 mm, distribute the fragments evenly over the dish, cultivate during 2 days without media replacement. While fragments attach and lie prone on the surface of the dish treated with CELLstart™ during these 2 days lots of cells will migrate to periphery of the cell clusters and get the morphology similar to that of mesenchymal cells (“flat cells”). At the same time some part of cells evicted from the central part of attached cell cluster form secondary rosettes. To prepare 24 well plate treat the wells with CELLstart™, than add 0.5 ml of StemPro® NSC SFM medium for neural proliferation (see 1.3.) supplemented with growth factors (20 ng/ml of each bFGF and EGF) and place in the incubator. Remove flat cells from 35 mm dishes with secondary rosettes. Use 200 μl plastic tip to scratch all undesirable cells. Rinse the dishes with 1-2 ml of warm D-PBS without Ca²⁺ [symbol] Mg²⁺, add fresh warm medium DN2. Using syringe needle cut the fields with secondary rosettes, and transfer them in the minimal volume of the medium into the 0.5 ml microcentrifuge tube containing 0.1 ml of warm StemPro® Accutase®. Place no more than 10 fragments with secondary rosettes per one tube. Incubate during 3-4 minutes at room temperature, then pipette gentle about 100 times with 200 μl tip. The total time for cells in StemPro® Accutase® shouldn't exceed 10 minutes. Add 0.4 ml of warm StemPro® NSC SFM medium per each vial. Centrifuge the tubes for 4 minutes at 120-130 g, then aspirate as much supernatant as possible carefully without disturbing of the cell pellet. Transfer 250 ul of medium from the well of prepared 24 well plate into the tube with cell pellet, resuspend the cell pellet using 200 ul tip, and transfer the cell suspension back into the well. Transfer the cells from one tube into the single well. Cell clones obtained by this way are considered as passage 0. During following 4-6 days observe cell proliferation, mark the wells that contain plenty cells similar to NSCs morphologically, i.e. of specific angular shape, capable to form small rosette-like structures (asterisks), capable to differentiate spontaneously into neuron-like cells in the fields with low density of the cells. During first 2-5 passages maintain the cells in the wells of the 24 well plate, split in the ratio 1:2 or 1:1. Discard those wells, in which cells start losing specific morphology. After obtaining of sufficient amount of cells, cultivate them on 35 mm Petri dishes, then on 60 mm Petri dishes.

### Example 7

### Differentiation of phNSC

Spontaneous differentiation of NSC was performed in the Neurobasal medium (Invitrogen), supplemented with B27 without retinol (Invitrogen), 0.1 mM MEM nonessential amino acids, 2 mM L-glutamine (GlutaMAX-I, Invitrogen) and antibiotic solution.

In the B27 supplemented medium without growth factors bFGF and EGF, spontaneous differentiation of phNSC and hNSC occurred within 4 week resulting in the generation of predominantly neuron-like cells Immunocytochemical analysis revealed the presence of neuron-specific tubulin βIII in the phNSC derivatives. Transcriptional activity analysis revealed neuronal specific markers TUBB3 (tubulin βIII) and MAP2, as well as the astrocyte marker GFAP in both phNSC and hNSC derivatives (FIG. 3). At the same time, the expression of oligodendrocyte marker FOXO4 was higher in the differentiated from phNSC cell population.

### Example 8

### phNSC Maintenance and Phenotype

The result of isolation and NEP rosettes dissociation to single cell suspension was the proliferating cells population; in general these cells were passaged every 4-5 days at split ratio 1:2 over 4.5 months. During at least 27 passages phNSC maintained specific morphology, similar to hNSC.

Specifically, maintaining and passaging of NSCs is performed as described in GIBCO-Invitrogen User Manual (MAN0001758) with some modifications.

NSCs are passaged once every 3-5 days at 1:2 split ratio depending on proliferation rate. Seeding density should be at least 5×10⁴ per cm², as cells tend to differentiate at low density.

Cells are ready to be passaged, when they form loose monolayer. Overgrowth of cells and dense monolayer can lead to differentiation and loss of subline. To passage the cells:
Prepare needed amount of 60 mm Petri dishes, treated with CELLstart™ 2-3 hours before passaging;
Warm needed amount of StemPro® NSC SFM medium, 6 ml of medium per one 60 mm Petri dish in the incubator at +37° C., 5% CO₂, humidified atmosphere; Prepare 15 ml centrifuge tubes with 10 ml of StemPro® NSC SFM medium, each tube per 1-2 60 mm Petri dishes. Place the tubes in the incubator at +37° C., 5% CO₂, humidified atmosphere;
Add needed amount of StemPro® Accutase® in the centrifuge tube, 1 ml per each dish to be passaged. Warm it in the water bath 20-30 minutes before passaging; 2-3 hours after adding of CELLstart™ add growth factors EGF and bFGF to the final concentration of 20 ng/ml of both;
Replace CELLstart™ solution with 6 ml of the StemPro® NSC SFM medium, supplemented with growth factors, place the dishes back to the incubator;
Take the dish to be passaged, tubes with StemPro® Accutase® and StemPro® NSC SFM medium without growth factors;
Aspirate the medium prom the dish and add 1 ml of warm StemPro® Accutase®, incubate at room temperature for 4 minutes. Shake the dish carefully during incubation;
After 4 minutes check under the inverted microscope if the cells start detaching from the surface. If not, additional incubation is needed, place the dish in the incubator for 1 minute;

Add 1 ml of StemPro® NSC SFM medium without growth factors to the dish and gently pipet cell suspension with 1000 ul tip to detach the cells;
Transfer the cell suspension to the tube containing StemPro® NSC SFM medium without growth factors. One tube fits up to 2 60 mm Petri dishes;
Carefully shake the tube to mix the contents;
Centrifuge the cells at 120-130 g for 4 minutes, carefully aspirate all the supernatant;
Take needed amount of new CELLstart™ treated dishes. Usually cells are passaged 1:2, so if there are cells from one 60 mm Petri dish, use 2 new dishes;
Transfer 1 ml of medium from each dish to the centrifuge tube to resuspend the pellet. Transfer 1 ml of cell suspension back to each of the dishes.
Distribute the cells equally in the dish by shaking it. Place the dishes in the incubator at +37° C., 5% CO₂, humidified atmosphere.

On the next day after passaging replace the culture medium with the fresh one. Later the medium should be replaced at least once every other day.

Transcriptional activity qRT-PCR analysis revealed the expression levels of SOX2, NES, MS1 and PAX6 in phNSC close to those in hNSC. The expression of OCT4 was at detectable but very low levels either in phNSC or in hNSC; endodermal marker AFP was not detected in all NSC lines (data not shown). Transcriptional activity levels of genes FOXD3 and SNAI2 specific for neural crest ectomesenchyme as well as mesodermal marker ACTA1 were lower in phNSC compared with hNSC, whereas a high level of neural tube neurogenic domain marker OLIG2 expression was revealed in phNSC. SOX2, NES and MS1 expression was also confirmed at the protein level by immunocytochemistry. Surface markers CD133 and CD15 analysis revealed that phNSC represent mixed population of positive and negative CD133 and CD15 cells (data not shown)

### Example 9

### Production of Parthenogenetically Derived Dopminergic Neurons

Multipotent neural precursor cells (NPC) have been derived from neuroectoderm which was derived from parthenogenetic stem cells either homozygous or heterozygous. The parthenogenetically derived NPCs differentiate into neurons such as midbrain dopaminergic neurons (DA). These DA neurons exhibit a midbrain phenotype and express TH, GIRK2, PITX3, NURR1, LMXA1, and EN1 as measured by immunocytochemistry and RT-PCR. As it is known from prior art, the main function of dopaminergic neurons is to release dopamine. Dopamine's major function in the body is reward-driven learning. The DA neurons derived from hpNPC also release dopamine as determined by LC/MS/MS. Whole cell electrophysiology proved that the parthenogenetically derived dopaminergic neurons are capable of firing action potentials.

Determination of Dopamine Release by LC/MS/MS

Analyzed dopamine levels in culture by LC-MS/MS using a 2.1×100 mm Atlantis-dC18 2.1×100 mm column (Waters). Conditioned medium from cultures of stem cell-derived dopaminergic neurons was collected, supplemented with 1 mM EDTA, and frozen at −80° C. Samples were thawed at room temperature and 200 μL of sample and standards were mixed with 500 μL of complexing agent 0.2% DPBA-ethanolamine ester+5 g/L EDTA in 2M NH₄Cl—NH₄OH, pH8.5. Oasis HLB micro-elution plates 30 μm (Waters) were conditioned with 0.5 mL methanol followed by 0.5 mL 0.2M NH₄Cl—NH₄OH pH 8.5. Complexed samples and standards were extracted slowly at a rate <0.5 mL/min in conditioned Oasis HLB micro-elution plates. The extraction plates were washed with 0.5 mL of 0.2M NH₄Cl—NH₄OH pH 8.5 followed by 0.5 mL of 20:80 methanol: 0.2M NH₄Cl—NH₄OH, pH 8.5. Dopamine was then eluted with 100 μL of 4% formic acid in water and collected into 96-well plate. 20 μL were loaded into Atlantis DC-18 2.1×100 mm i.d. column and separation of the injected samples was achieved by gradient elution in 0.1% formic acid-acetonitrile mobile phase at a flow rate 300 μL/min for 8 minutes. Peaks were analyzed with a PE SCIEX API 4000 LC/MS/MS mass spectrometer (SpectraLab Scientific Inc.) and quantified by Multiple Reaction Monittoring (MRM).

The DA neurons derived from hpNPC also release dopamine as determined by LC/MS/MS. Sample #5 is dopamine level released by DA neurons derived from phNSC. (Table 6)

TABLE 6

| | Concentration |
|---|---|
| Sample ID | (ng/mL) |
| Sample 1 | BLQ |
| Sample 2 | 12.2 |
| Sample 3 | BLQ |
| Sample 4 | 0.864 |
| Sample 5 | 2.15 |
| Sample 6 | BLQ |

BLQ Below Limit of Quantitation

Electrophysiology

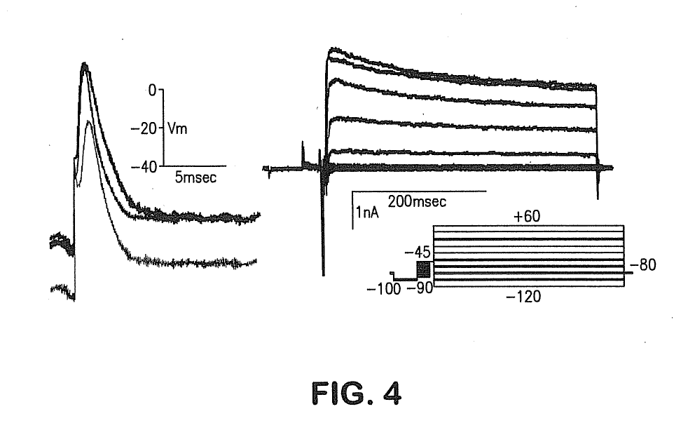

The coverslips where the neurons were growing were cut into smaller segments to fit into the fusiform test chamber sized 5 mm at the widest point and 1 cm long. The test chamber was perfused with Tyrodes solution containing 1.8 mM CaCl2; 1 mM MgCl2; 4 mM KCl; 140 mM NaCl; 10 mM glucose; 10 mM HEPES; 305-315 mOsm; pH 7.4 (adjusted with 5 M NaOH). Electrodes were prepared with 3-5 MOhms resistance when filled with 140 mM KCl; 10 mM MgCl2; 6 mM EGTA; 5 mM HEPES-Na; 5 mM ATP-Mg; 295-305 mOsm; pH 7.25 (adjusted with 1 M KOH). Data were processed with a 5 KHz Bessel filter and acquired at 10-20 KHz using a Multiclamp 700 A amplifier (Axon Intruments) and Pclamp software. All experiments were performed at room temperature under a microscope in a continuous flow chamber. Whole cell electrophysiology proved that the parthenogenetically derived dopaminergic neurons are capable of firing action potentials. Data is shown in FIG. 4.

### REFERENCES

1. ES. Revazova, N. A. Turovets, O. D. Kochetkova, L. B. Kindarova, L. N. Kuzmichev, J. D. Janus, M. V. Pryzhkova, Patient-specific stem cell lines derived from human parthenogenetic blastocysts, Cloning Stem Cells 9 (2007) 432-449.
2. E. S. Revazova, N. A. Turovets, O. D. Kochetkova, L. S. Agapova, J. L. Sebastian, M. V. Pryzhkova, V. I. Smolnikova, L. N. Kuzmichev, J. D. Janus, HLA homozygous stem cell lines derived from human parthenogenetic blastocysts, Cloning Stem Cells 10 (2008) 11-24.
3. C. J. Taylor, E. M. Bolton, S. Pocock, L. D. Sharples, R. A. Pedersen, J. A. Bradley, Banking on human embryonic stem cells: estimating the number of donor cell lines needed for HLA matching, Lancet 366 (2005) 2019-2025.
4. B. A. Reynolds, S. Weiss, Generation of neurons and astrocytes from isolated cells of the adult mammalian central nervous system, Science 225 (1992) 1707-1710.
5. H. Okano, Neural stem cells and strategies for the regeneration of the central nervous system, Proc. Jpn Acad. Se.r B Phys. Biol. Sci. 86 (2010) 438-450.
6. S. Eckardt, T. C. Dinger, S. Kurosaka, N. A. Leu, A. M. Müller, K. J. McLaughlin, In vivo and in vitro differentiation of uniparental embryonic stem cells into hematopoietic and neural cell types, Organogenesis 4 (2008) 33-41.
7. J. B. Cibelli, K. A. Grant, K. B. Chapman, K. Cunniff, T. Worst, H. L. Green, S. L. Walker, P. H. Gutin, L. Vilner, V. Tabar, T. Dominko, J. Kane, P. J. Wettstein, R. P. Lanza, L. Studer, K. E. Vrana, M. D. West, Parthenogenetic stem cells in nonhuman primates, Science, 295 (2002) 819.
8. J. V. Harness, N. A. Turovets, M. J. Seiler, G. Nistor, G. Altun, L. S. Agapova, D. Ferguson, L. C. Laurent, J. F. Loring, H. S. Keirstead, Equivalence of conventionally-derived and parthenote-derived human embryonic stem cells, PLoS One. 6 (2011) e14499.
9. A. Nagy, M. Sass, M. Markkula, Systematic non-uniform distribution of parthenogenetic cells in adult mouse chimaeras, Development 106 (1989) 321-324.
10. R. Sánchez-Pernaute, L. Studer, D. Ferrari, A. Perrier, H. Lee, A. Viñuela, 0. Isacson, Long-term survival of dopamine neurons derived from parthenogenetic primate embryonic stem cells (cyno-1) after transplantation, Brain. 128 (2005) 1498-1510.
11. S. Shin, M. Mitalipova, S. Noggle, D. Tibbitts, A. Venable, R. Rao, S. L. Stice, Long-term proliferation of human embryonic stem cell-derived neuroepithelial cells using defined adherent culture conditions, Stem Cells 24 (2006) 125-138.
12. S. K. Dhara, S. L. Stice, Neural differentiation of human embryonic stem cells, J. Cell Biochem. 105 (2008) 633-640.
13. T. C. Archer, J. Jin, E. S. Casey, Interaction of Sox1, Sox2, Sox3 and Oct4 during primary neurogenesis, Dev. Biol. 350 (2011) 429-440.
14. V. Graham, J. Khudyakov, P. Ellis, L. Pevny, SOX2 functions to maintain neural progenitor identity, Neuron 39 (2003) 749-765.
15. Y. Sun, W. Kong, A. Falk, J. Hu, L. Zhou, S. Pollard, A. Smith, CD133 (Prominin) negative human neural stem cells are clonogenic and tripotent, PLoS One 4 (2009) e5498.
16. S. Colleoni, C. Galli, S. G. Giannelli, M. T. Armentero, F. Blandini, V. Broccoli, G. Lazzari, Long-term culture and differentiation of CNS precursors derived from anterior human neural rosettes following exposure to ventralizing factors, Exp. Cell Res. 316 (2010) 1148-1158.
17. T. Brevini and F. Gamdolfi, Parthenotes as a Source of Embryonic Stem Cells, Cell Prolif. 41 (2008) 20-30.
18. Z Kastenberg and J. Odorico, Alternative Sources of Pluripotency: Scienc, Ethics and Stem Cells, Transplant. Rev. 22 (2008) 215-222.
19. K. Kim, K. Ng, P. Rugg-Gunn, J. Shish, 0. Kirak, R Jaenisch, T. Wakayama, M. Moore, R. Pedersen and G. Daley, Recombination Signatures Distinguish Embryonic Stem Cells Derived by Parthenogenesis and Somatic Cell Nuclear Transfer, Cell Stem Cell 1 (2007) 1-7.
20. M. Amit, M. Carpenter, M. Inokurna, C. Chiu, C. Harris, M. Waknitz, J. Itskovitz-Eldor, and J. Thomson, Clonally derived human embryonic stem cell lines maintain pluripotency and proliferative potential for prolonged periods of culture, Dev. Biol (2000) 227:271-278.
21. M. Amit, C. Shariki, V. Margulets, and J. Itskovitz-Eldor, Feeder layer- and serum-free culture of human embryonic stem cells, Biol Reprod (2004) 70:837-845.
22. D. Hoffman and M. Carpenter, Characterization and culture of human embryonic stem cells, Nature Biotech (2005) 23(6):699-708.
23. S. Kim, S. Cheon, S. Yoo, J. Kwon, J. Park, C. Kim, K. Rhee, S. You, J. Lee, S. Roh, H. Yoon, Contribution of the PI3K/Akt/PKB signal pathway to maintenance of self-renewal in human embryonic stem cells, FEBS Lett (2005) 579:534-540.
24. N. Sato, L. Meijer, L. Skaltsounis, P. Greengard, and A. Brivanlou, Maintenance of pluripotency in human and mouse embryonic stem cells through activation of Wnt signaling by a pharmacological GSK-3-specific inhibitor, Nat Med (2004) 10:55-63.
25. R. Palacios, E. Golunski, and J. Samaridis, In vitro generation of hematopoietic stem cells from an embryonic stem cell line, Proc. Natl Acad. Sci., 92 (1995) 7530-7537.
26. J. Pedersen, Studies of in vitro differentiation with embryonic stem cells, Reprod. Fertil. Dev. (1994) 6:543-552.
27. G. Bain, D. Kitchens, M. Yao, J. Huettner, and D. Gottlieb, Embryonic stem cells express neuronal properties in vitro, Dev. Biol. (1995) 168:342-357.

Although the invention has been described with reference to the above example, it will be understood that modifications and variations are encompassed within the spirit and scope of the invention. Accordingly, the invention is limited only by the following claims.

What is claimed is:

1. An isolated neuronal stem cell, wherein the cell is differentiated from a parthenogenetically activated oocyte.

2. The neuronal stem cell of claim 1, wherein the cell is histocompatible with the oocyte donor.

3. The neuronal stem cell of claim 2, wherein the cell is histocompatible with a population group based on a matching haplotype.

4. The neuronal stem cell of claim 1, wherein the cell has a different pattern of zygosity from an ESC.

5. The neuronal stem cell of claim 1, wherein the cell contains only the maternal genome.

6. The neuronal stem cell of claim 1, wherein the cell is histocompatible with the oocyte donor, has a different pattern of zygosity from an ESC and contains only the maternal genome.

7. The neuronal stem cell of claim 1, wherein the cell is transplantable to humans.

8. The neuronal stem cell of claim 1, wherein the cell is undifferentiated, partially differentiated or fully differentiated.

9. The neuronal stem cell of claim 1, wherein the cell can be differentiated into a neuronal cell.

10. The neuronal stem cell of claim 9, wherein the cell can be differentiated a neuronal cell selected from the group consisting of a neuron, a glial cell, an oligodendrocyte and an astrocyte.

11. The differentiated neuronal cell of claim 10, wherein the cell is a neuron.

12. The neuron of claim 11, wherein the neuron is selected from the group consisting of: a cholinergic neuron, a GABAergic neuron, a glutamatergic neuron, a dopaminergic neuron and a serotonergic neuron.

13. The neuron of claim 12, wherein the neuron is a dopaminergic neuron.

14. The neuronal stem cell of claim 1, wherein the cell express neural markers selected from the group consisting of: SOX2, Nestin, Mushashi-1, TUBB3, MAP2, FOXO4, GFAP, CD113 and CD15

15. A method for producing a neuronal stem cells by differentiating parthenogenetically derived human stem cells, the method comprising:
a) growing parthenogenetically derived human stem cells on a feeder layer of fibroblast cells for at least 2 days;
b) growing parthenogenetically derived human stem cells on a petri dish without fibroblast feeder layer for at least 1 day;
c) culturing the cells in a neuronal induction media;
d) obtaining a single cell suspension of the cells from (c); and
e) culturing the single cells from step (d) on a petri dish with no fibroblast feeder layer in a neuronal proliferation media.

16. The method of claim 15, wherein the neuronal induction media comprises:
a) Penicillin-Streptomycin-Amphotericin Solution
b) DMEM/F12;
c) MEM Non-Essential Amino Acids Solution;
d) L-Glutamine;
e) N2 Supplement; and
f) bFGF.

17. The method of claim 15, wherein the neuronal proliferation media comprises:
a) Penicillin-Streptomycin-Amphotericin;
b) DMEM/F12;
c) GlutaMAX™-I;
d) StemPro® Neural Supplement;
e) bFGF; and
f) EGF.

18. The method of claim 15, wherein the petri dish is coated with CELLstart.

19. A neuronal stem cell produced by the method of claim 15.

20. The method of claim 15, wherein a neuroepithelial rosette forms in about 1-2 weeks.

21. Isolated neuronal stem cells derived from parthenogenetically derived human stem cells using the method comprising:
a) growing parthenogenetically derived human stem cells on a feeder layer of fibroblast cells for at least 2 days;
b) growing parthenogenetically derived human stem cells on a petri dish with no fibroblast feeder layer for at least 1 day;
c) culturing the cells in a neuronal induction media;
d) obtaining a single cell suspension of the cells from (c); and
e) culturing the single cells from step (d) on a petri dish with no fibroblast feeder layer in a neuronal proliferation media.

22. The cells of claim 21, wherein the cells express neural markers selected from the group consisting of: SOX2, Nestin, Mushashi-1, TUBB3, MAP2, FOXO4, GFAP, CD113 and CD15.

23. The cells of claim 21, wherein the neuronal stem cells maintain the neuronal phenotype for at least 27 passages.

24. The cells of claim 21, wherein the neuronal stem cells can differentiate into neuronal cells.

25. The cells of claim 24, wherein the neuronal cells are selected from the group consisting of neurons, glial cells, astrocytes and oligodendrocytes.

26. The differentiated neuronal cell of claim 25, wherein the cell is a neuron.

27. The neuron of claim 26, wherein the neuron is selected from the group consisting of: a cholinergic neuron, a GABAergic neuron, a glutamatergic neuron, a dopaminergic neuron and a serotonergic neuron.

28. The neuron of claim 27, wherein the neuron is a dopaminergic neuron.

29. The cells of claim 21, wherein the cell is histocompatible with the oocyte donor.

30. The cells of claim 21, wherein the cell has a different pattern of zygosity from an ESC.

31. The cells of claim 21, wherein the cell contains only the maternal genome.

32. The neuronal stem cell of claim 21, wherein the cell is histocompatible with the oocyte donor, has a different pattern of zygosity from an ESC and contains only the maternal genome.

33. The neuronal stem cell of claim 21, wherein the cell is transplantable to humans.

34. A method of treating a neurologic disorder using neuronal stem cells produced from parthenogenetically derived from oocytes.

35. The method of claim 34, wherein the neurologic disorder is selected from the group consisting of: epilepsy, convulsions, neurotoxic injury, hypoxia, anoxia, ischemia, stroke, cerebrovascular accident, brain or spinal cord trauma, myocardial infarct, physical trauma, drowning, suffocation, perinatal asphyxia, hypoglycemic events, neurodegeneration, Alzheimer's disease, senile dementia, Amyotrophic Lateral Sclerosis, Multiple Sclerosis, Parkinson's disease, Huntington's disease, Down's Syndrome, Korsakoff's disease, schizophrenia, AIDS dementia, multi-infarct dementia, Binswanger dementia, neuronal damage, seizures, chemical toxicity, addiction, morphine tolerance, opiate tolerance, opioid tolerance, barbiturate tolerance, acute and chronic pain, migraine, anxiety, major depression, manic-depressive illness, obsessive-compulsive disorder, schizophrenia and mood disorders, bipolar disorder, unipolar depression, dysthymia, seasonal effective disorder, dystonia or other movement disorders, sleep disorder, muscle relaxation and urinary incontinence.

36. The method of claim 34, wherein the neuronal stem cells are implanted into a patient in need of such treatment.

37. A method of differentiating neuronal stem cells, the method comprising culturing neuronal stem cells in neuronal differentiation media.

38. The method of claim 37, wherein the neuronal differentiation media comprises:
a) Penicillin-Streptomycin-Amphotericin;
b) DMEM/F12;
c) GlutaMAX™-I; and
d) StemPro® Neural Supplement.

39. The method of claim 37 wherein the neuronal stem cells are differentiated into a neuronal cell selected from the group consisting of: a neuron, a glial cell, an oligodendrocyte and an astrocyte.

40. The differentiated neuronal cell of claim 39, wherein the cell is a neuron.

41. The neuron of claim 40, wherein the neuron is selected from the group consisting of: a cholinergic neuron, a GABAergic neuron, a glutamatergic neuron, a dopaminergic neuron and a serotonergic neuron.

42. The neuron of claim 41, wherein the neuron is a dopaminergic neuron.

43. Differentiated cells produced by the method of claim 37.

44. The cells of claim 37, wherein the cells are differentiated from a parthenogenetically activated oocyte.

45. A method for producing neuronal stem cells by differentiating parthenogenetically derived human stem cells, the method comprising:
a) cultivation of human pluripotent stem cells in feeder-free conditions;
b) exposure of said cells to neuronal induction medium;
c) mechanical isolation of partially differentiated cells; and
d) further expansion and maintenance of said cells until maturation.

46. The method of claim 45, wherein the neuronal induction media comprises:
a) Penicillin-Streptomycin-Amphotericin Solution;
b) DMEM/F12;
c) MEM Non-Essential Amino Acids Solution;
d) L-Glutamine;
e) N2 Supplement; and
f) bFGF.

47. The method of claim 45, wherein the neuronal proliferation media comprises:
a) Penicillin-Streptomycin-Amphotericin;
b) DMEM/F12;
c) GlutaMAX™-I;
d) StemPro® Neural Supplement;
e) bFGF; and
f) EGF.

48. The method of claim 45, wherein feeder-free conditions utilize the ECM substrate including but not limited to: CELLstart, Matrigel, laminin, gelatin, fibronectin.

49. The neuronal stem cell of claim 45.

50. The method of claim 45, wherein a neuroepithelial rosette forms after 1-2 weeks.

51. Isolate neuronal stem cells derived from parthenogenetically derived human stem cells using the method comprising:
a) cultivation of human pluripotent stem cells in feeder-free conditions;
b) exposure of said cells to neuronal induction medium;
c) mechanical isolation of partially differentiated cells; and
d) further expansion and maintenance of said cells until maturation.

52. The cells of claim 51, wherein the cells express neural stem cell markers selected from the group consisting of: SOXB1-family NES, MSH-1, CXCR4, CCND1, LHX2, PAX6 and GAP43.

53. The cells of claim 51, wherein the neuronal stem cells maintain the neuronal phenotype for at least 30 passages.

54. The cells of claim 51, wherein the neuronal stem cells can differentiate into neuronal cells.

55. The cells of claim 54, wherein the neuronal cells are selected from the group consisting of neurons, astrocytes and oligodendrocytes.

56. A method of treating a neurologic disorder using neuronal stem cells derived from parthenogenetically derived from oocytes.

57. The method of claim 56, wherein the neurologic disorder is selected from the group consisting of: epilepsy, convulsions, neurotoxic injury, ischemia, stroke, cerebrovascular accident, brain or spinal cord trauma, physical trauma, Alzheimer's disease, senile dementia, Amyotrophic Lateral Sclerosis, Multiple Sclerosis, Parkinson's disease, Huntington's disease, schizophrenia, neuronal damage, migraine, anxiety, major depression, manic-depressive illness, obsessive-compulsive disorder, schizophrenia and mood disorders, bipolar disorder, unipolar depression, dystonia or other movement disorders, sleep disorder, muscle relaxation.

58. The method of claim 56, wherein the neuronal stem cells are implanted into a patient in need of such treatment.

59. A method of differentiating neuronal stem cells, the method comprising culturing neuronal stem cells in neuronal differentiation media.

60. The method of claim 59, wherein the neuronal differentiation media comprises:
a) Penicillin-Streptomycin-Amphotericin;
b) DMEM/F12;
c) GlutaMAX™-I; and
d) StemPro® Neural Supplement.

61. The method of claim 59 wherein the neuronal stem cells are differentiated into a neuronal cell selected from the group consisting of: a neuron, an oligodendrocyte and an astrocyte.

62. The differentiated cells of claim 59.

63. The cells of claim 59, wherein the cells are differentiated from a parthenogenetically activated oocyte.
